(19) United States
(12) Patent Application Publication
(10) Pub. No.: US 2011/0212444 A1
(43) Pub. Date: Sep. 1, 2011

(54) USE OF METHYLATION STATUS OF MINT LOCI AND TUMOR RELATED GENES AS A MARKER FOR MELANOMA AND BREAST CANCER

(76) Inventors: Dave S.B. Hoon, Los Angeles, CA (US); Atsushi Tanemura, Osaka (JP); Anneke van Hoesel, Santa Monica, CA (US)

(21) Appl. No.: 12/981,469

(22) Filed: Dec. 29, 2010

Related U.S. Application Data
(62) Division of application No. 12/344,179, filed on Dec. 24, 2008.
(60) Provisional application No. 61/017,493, filed on Dec. 28, 2007.

(51) Int. Cl. C12Q 1/68

(52) U.S. Cl. 435/6.11

(57) ABSTRACT

The invention relates to a method of detecting melanoma or breast cancer using DNA methylation in MINT17, MINT31, or the promoter region of WIF1, TFPI2, RASSF1A, SOCS1, GATA4, or RARβ2 as a biomarker. Also disclosed are methods of using the biomarker for determining the cancer status and predicting the outcome of the cancer.

### RELATED APPLICATION

This application is a divisional application of U.S. application Ser. No. 12/344,179, filed Dec. 24, 2008, which claims priority to U.S. Provisional Application Ser. No. 61/017,493, filed on Dec. 28, 2007, the content of which is incorporated herein by reference in its entirety.

### FUNDING

This invention was made with support in part by grants from NIH, NCI Project II P0 CA029605 and CA012582 grants. Therefore, the U.S. government has certain rights.

### FIELD OF THE INVENTION

The present invention relates in general to the MINT (methylated-in-tumor) loci and TRGs (tumor-related genes). More specifically, the invention relates to the use of the methylation status of some specific MINT loci and TRGs as a diagnostic, prognostic, and predictive biomarker in the management of melanoma and breast cancer.

### BACKGROUND OF THE INVENTION

Cutaneous malignant melanoma is the sixth most common cancer in the United States and a major public health problem worldwide for which survival depends on both early detection and eradication of disease (1). To date, there have been limited studies addressing the role of epigenetic changes during early tumor progression, or evaluating differences in the epigenetic patterns of primary versus metastatic tumors. However, epigenetic inactivation of tumor suppressor genes has been implicated in tumorigenesis and progression of a variety of different malignancies (2-4), and recent studies are beginning to demonstrate the role of epigenetic events in cutaneous melanoma (5, 6). Existing prognostic factors for primary melanoma include Breslow thickness and ulceration, but the clinical utility of these pathologic characteristics is limited. Delineation of factors involved in the progression of primary tumors may aid in the identification of individuals at high risk for recurrence, and may guide the development of future targeted treatment strategies for patients with high-risk resected or metastatic disease.

While the observation of methylation changes in CpG island promoter regions in a few tumor-related and -suppressor genes has been reported in the case of malignant cutaneous melanoma (7, 8), the clinical significance of these molecular aberrations is still being defined. For example, it has been well demonstrated in other tumor systems that tissue factor pathway inhibitor-2 (TFPI2) inhibits tumor growth, invasion, metastasis and angiogenesis, and induces apoptosis (9). Nobeyama et al. (10) noted that TFPI2 was methylated in 5 of 17 (29%) metastatic melanoma lesions but none of the primary tumors examined, suggesting that methylation-induced inactivation of this gene is involved in melanoma metastasis. Silencing of WIF1, a Wnt pathway antagonist, has been implicated in cellular proliferation of a variety of tumor types including non-small cell lung cancer (11), bladder and renal cell cancers (12, 13) and gastrointestinal cancers (14), and restoration of WIF1 expression has been shown to inhibit growth of melanoma in vitro and in vivo (15). SOCS1 is a known tumor suppressor gene that has been found circulating in the methylated form in melanoma patients (7). Expression of GATA4, a gene encoding a transcription factor thought to act like a tumor suppressor gene through its activation of several other genes with antitumor effects, has been found to be epigenetically silenced in gastrointestinal cancers (16) and lung cancer (17), although there are no reports to date of its role in melanoma development. RARβ2 methylation has been previously shown to be present in a high percentage of clinical melanoma specimens, and to be associated with increased Breslow depth of primary melanomas (5) implicating its role in tumorigenesis. The significance of RASSF1A and RARβ2 hypermethylation in predicting non-responsiveness to biochemotherapy in AJCC stage IV melanoma patients has been demonstrated (6).

In gastric and colorectal cancer, the existence of a CpG island methylator phenotype (CIMP) has been described, and found to be associated with tumor development through coordinated inactivation of multiple tumor suppressor and mismatch repair genes (4, 18). The CIMP is marked by methylation of multiple non-coding, methylated-in-tumor (MINT) loci, which have been shown to underlie epigenetic changes in gastrointestinal tumors. Methylation of MINT loci is thought to be associated with a high degree of hypermethylation of tumor-related genes (TRGs), as observed for example with the high prevalence of p16 and THBS1 hypermethylation in CIMP+ colorectal tumors (18). The CIMP has also been shown to be a predictive marker of survival benefit from adjuvant 5-FU-based chemotherapy in patients with colorectal carcinoma metastatic to regional lymph nodes (19).

### SUMMARY OF THE INVENTION

The present invention is based, at least in part, upon the unexpected discovery that the methylation status of MINT (methylated-in-tumor) 17, MINT31, and the promoter regions of TFPI2 (tissue factor pathway inhibitor-2), WIF1 (Wnt inhibitory factor-1), SOCS1 (suppressor of cytokine signaling-1), RASSF1A (Ras association domain family 1A), GATA4 (GATA binding protein 4), and RARβ2 (retinoic acid receptor beta 2) can be used as a biomarker for diagnosis and prognosis of melanoma and breast cancer.

Accordingly, in one aspect, the invention features a method of determining melanoma status. The method comprises providing from a subject a sample containing melanoma cells and determining the level of DNA methylation in MINT17, MINT31, or the promoter region of WIF1, TFPI2, RASSF1A, or SOCS1 in the melanoma cells. The level of methylation in MINT17, MINT31, or the promoter region of WIF1, TFPI2, RASSF1A, or SOCS1 in the melanoma cells, if higher than that in normal cells, indicates that the melanoma is likely to be an aggressive melanoma.

For example, the level of DNA methylation in the promoter region of TFPI2 in the melanoma cells indicates that the subject is suffering from AJCC (American Joint Committee on Cancer) Stage II, III, or IV melanoma; the level of DNA methylation in the promoter region of RASSF1A in the melanoma cells indicates that the subject is suffering from AJCC Stage III or IV melanoma; and a higher level of DNA methylation in MINT31 or the promoter region of WIF1, TFPI2, RASSF1A, or SOCS1 in the melanoma cells indicates that the subject is suffering from a more aggressive melanoma.

In some embodiments, the subject is suffering from an AJCC Stage I, II, or III melanoma, and a higher level of DNA methylation in MINT17 in the melanoma cells indicates that the subject is suffering from a more aggressive melanoma. In some embodiments, the subject is suffering from an AJCC Stage I, II, or IV melanoma, and a higher level of DNA methylation in MINT17 in the melanoma cells indicates that the subject is suffering from a more aggressive melanoma.

In another aspect, the invention features a method of predicting the outcome of melanoma. The method comprises providing from a subject a sample containing melanoma cells and determining the level of DNA methylation in MINT31 in the melanoma cells. A higher level of methylation in MINT31 in the melanoma cells indicates a more likelihood of disease-free survival and overall survival.

In some embodiments, the subject is suffering from an AJCC Stage III melanoma.

Also within the invention is a method of detecting melanoma in a subject. The method comprises providing a DNA sample from a subject and determining the level of DNA methylation in MINT17, MINT31, or the promoter region of GATA4 in the sample. The level of DNA methylation in MINT17, MINT31, or the promoter region of GATA4 in the sample, if higher than that in normal cells, indicates that the subject is suffering from melanoma such as an aggressive melanoma.

The invention further provides a method of determining breast cancer status. The method comprises providing from a subject a sample containing breast cancer cells and determining the level of DNA methylation in MINT17, MINT31, or the promoter region of RASSF1A or RARβ2 in the breast cancer cells. The level of DNA methylation in MINT17, MINT31, or the promoter region of RASSF1A or RARβ2 in the breast cancer cells, if higher than that in normal cells, indicates that the breast cancer is likely to be an aggressive breast cancer. The breast cancer may be a primary or metastatic cancer.

For example, a higher level of DNA methylation in MINT17 or the promoter region of RASSF1A, or a lower level of DNA methylation in MINT31 in the breast cancer cells indicates that the breast cancer is more likely to be positive for ER (estrogen receptor); a higher level of DNA methylation in MINT17 or the promoter region of RASSF1A, or a lower level of DNA methylation in MINT31 or the promoter region of RARβ2 in the breast cancer cells indicates that the breast cancer is more likely to be positive for PR (progesterone receptor); a higher level of DNA methylation in the promoter region of RARβ2, or a lower level of DNA methylation in MINT17 in the breast cancer cells indicates that the breast cancer is more likely to be positive for HER2 (human epidermal growth factor receptor 2); and a higher level of DNA methylation in MINT31 in the breast cancer cells indicates that the subject is likely to be suffering from a more aggressive breast cancer.

The invention also provides a method of predicting the outcome of breast cancer. The method comprises providing from a subject a sample containing breast cancer cells and determining the level of DNA methylation in MINT17, MINT31, or the promoter region of RARβ2 in the breast cancer cells. A higher level of methylation in MINT17, MINT31, or the promoter region of RARβ2 in the breast cancer cells indicates a less likelihood of overall survival.

In addition, the invention provides a method of detecting breast cancer in a subject. The method comprises providing a DNA sample from a subject and determining the level of DNA methylation in MINT17 or MINT31 in the sample. The level of DNA methylation in MINT17 or MINT31 in the sample, if higher than that in normal cells, indicates that the subject is suffering from breast cancer such as an aggressive breast cancer. The breast cancer may be a primary or metastatic cancer.

Unless otherwise defined, all technical and scientific terms used herein have the same meaning as commonly understood by one of ordinary skill in the art to which this invention pertains. In case of conflict, the present document, including definitions, will control. The materials, methods, and examples disclosed herein are illustrative only and not intended to be limiting. Other features, objects, and advantages of the invention will be apparent from the description and the accompanying drawings, and from the claims.

### BRIEF DESCRIPTION OF THE FIGURES

FIG. 1. Methylation of MINT loci Increases with Advancing AJCC Stage in Melanoma. The MINT17 (A) and MINT31 (B) methylation indices are shown for each tumor specimen stratified by AJCC stage. Horizontal bars represent mean values for grouping of each stage.

FIG. 2. Improved Disease Free and Overall Survival for AJCC Stage III Patients with MINT31 Methylation in Melanoma. Kaplan-Meier curves for disease-free (A) and overall survival (B) in stage III patients. The log-rank test confirmed improved disease-free (P=0.047) and overall survival (P=0.013) for patients with tumor samples with MINT31 methylation.

FIG. 3. AQAMA assay.

FIG. 4A. MINT17 MI for non-neoplastic vs. neoplastic breast tissue.

FIG. 4B. ROC curve for MINT17 as predictor for breast cancer.

FIG. 5. MINT17 MI vs. ER status in breast cancer patients.

FIG. 6. MINT17 MI vs. PR status in breast cancer patients.

FIG. 7. MINT 17 MI vs. S-phase fraction in breast cancer.

FIGS. 8A and 8B. Relation between Methylation Index and estrogen receptor status in breast cancer. Methylation Index of MINT17, MINT31 and RASSF1A are significantly correlated to ER status. In MINT17 and RASSF1A, higher MI is related to ER positivity. In MINT31, lower MI is related to ER positivity.

FIGS. 9A and 9B. Relation between Methylation Index and progesterone receptor status in breast cancer. Methylation Index of MINT17, MINT31, RARβ2, and RASSF1A are significantly correlated to PR status. In MINT17 and RASSF1A, higher MI is related to PR positivity. In MINT31 and RARβ2, lower MI is related to PR positivity.

FIGS. 10A and 10B. Relation between Methylation Index and HER2 status in breast cancer. Methylation Index of MINT17 and RARβ2 are significantly correlated to HER2 status. In MINT17, lower MI is related to HER2 positivity. In RARβ2, higher MI is related to HER2 positivity.

FIG. 11. Relation between Methylation Index and AJCC stage in breast cancer. Methylation Index of MINT31 is significantly correlated to AJCC stage. In MINT31, higher MI is related to stage progression.

FIG. 12. Correlation between methylation level of markers in breast cancer, parametric.

FIG. 13. Correlation between methylation level of markers in breast cancer, non-parametric.

FIGS. 14A and 14B. Overall disease related survival vs. MINT17 MI in breast cancer.

FIGS. 15A and 15B. Overall disease related survival vs. MINT31 MI in breast cancer.

FIGS. 16A and 16B. Overall disease related survival vs. RARβ2 MI in breast cancer.

FIG. 17. Overall disease related survival vs. RASSF1A MI in breast cancer.

### DETAILED DESCRIPTION OF THE INVENTION

The present invention relates to diagnosis and prognosis of melanoma and breast cancer using DNA methylation in MINT17, MINT31, or the promoter region of WIF1, TFPI2, RASSF1A, SOCS1, GATA4, or RARβ2 as a biomarker.

MINT17 and MINT31 loci are known in the art. A “promoter” is a region of DNA extending 150-300 bp upstream from the transcription start site that contains binding sites for RNA polymerase and a number of proteins that regulate the rate of transcription of the adjacent gene. The promoter regions of TFPI2, WIF1, SOCS1, RASSF1A, GATA4, and RARβ2 are also known in the art. For example, the human MINT17 and MINT31 loci and the promoter regions of TFPI2, WIF1, SOCS1, RASSF1A, GATA4, and RARβ2 are as follows:

Genebank Accession Numbers:

| | | |
|---|---|---|
| MINT17 | Chrom 12 | AF135517 |
| MINT31 | Chrom 17 | AF135531 |
| TFPI2 | Chrom 7q21.3 | NM_006528 |
| WIF1 | Chrom 12q14.3 | NM_007191 |
| SOCS1 | Chrom 16q13.13 | NM_003745 |
| RASSF1A | Chrom 3p21.31 | NM_170712 |
| GATA4 | Chrom 8p23.1 | NM_002052 |
| RARβ2 | Chrom 3p24.2 | NM_000965 |

As described in detail below, studies were designed to profile multiple tumor-related genes and members of the methylated-in-tumor (MINT) loci family to identify a CpG island methylator phenotype (CIMP) pattern in malignant cutaneous melanoma and breast cancer. The methylation status of CpG islands in the promoter region of six TRGs known to exhibit epigenetic aberrations associated with malignancy and seven MINT loci was examined to determine whether there exists a clinically significant CIMP related to melanoma or breast cancer progression. It was discovered that, in cutaneous melanoma and breast cancer, the CIMP is associated with tumor progression. In addition, several key tumor-related genes become progressively hypermethylated with the progression of the primary melanoma and breast cancer. By knowing the epigenetic biomarkers associated with advancing tumor stage, it is conceivable that their identification in primary tumors may help to identify those tumors at high-risk of metastasis or recurrence. Thus, the epigenetic biomarker phenotype of a primary melanoma or breast cancer could be used, in addition to currently utilized clinical and histopathologic features, to determine which patients may derive the most benefit from adjuvant therapy. Furthermore, the identification of epigenetic biomarkers may also be used to design future targeted therapeutics that act to reverse hypermethylation of selected tumor-related genes.

Accordingly, the invention first provides a method of detecting DNA methylation in melanoma cells. A sample containing melanoma cells is obtained from a cell culture or a subject. DNA methylation in MINT17, MINT31, or the promoter region of GATA4 is then detected in the melanoma cells.

Likewise, the invention provides a method of detecting DNA methylation in breast cancer cells. A sample containing breast cancer cells is obtained from a cell culture or a subject. DNA methylation in MINT17 or MINT31 is then detected in the breast cancer cells.

As used herein, a “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human. In another embodiment, the subject is an experimental animal or animal suitable as a disease model.

Methods for extracting cellular DNA are well known in the art. Typically, cells are lysed with detergents. After cell lysis, proteins are removed from DNA using various proteases. DNA is then extracted with phenol, precipitated in alcohol, and dissolved in an aqueous solution.

DNA methylation can be detected and quantified by any method commonly used in the art, for example, methylation-specific PCR (MSP), bisulfite sequencing, or pyrosequencing, and absolute quantitative analysis of methylated alleles (AQAMA).

MSP is a technique whereby DNA is amplified by PCR dependent upon the methylation state of the DNA. See, e.g., U.S. Pat. No. 6,017,704. Determination of the methylation state of a nucleic acid includes amplifying the nucleic acid by means of oligonucleotide primers that distinguish between methylated and unmethylated nucleic acids. MSP can rapidly assess the methylation status of virtually any group of CpG sites within a CpG island, independent of the use of methylation-sensitive restriction enzymes. This assay entails initial modification of DNA by sodium bisulfite, converting all unmethylated, but not methylated, cytosines to uracils, and subsequent amplification with primers specific for methylated versus unmethylated DNA. MSP requires only small quantities of DNA, is sensitive to 0.1% methylated alleles of a given CpG island locus. MSP eliminates the false positive results inherent to previous PCR-based approaches which relied on differential restriction enzyme cleavage to distinguish methylated from unmethylated DNA. This method is very simple and can be used on small amounts of samples. MSP product can be detected by gel electrophoresis, CAE (capillary array electrophoresis), or real-time quantitative PCR.

Bisulfite sequencing is widely used to detect 5-MeC (5-methylcytosine) in DNA, and provides a reliable way of detecting any methylated cytosine at single-molecule resolution in any sequence context. The process of bisulfite treatment exploits the different sensitivity of cytosine and 5-MeC to deamination by bisulfite under acidic conditions, in which cytosine undergoes conversion to uracil while 5-MeC remains unreactive.

Exemplary AQAMA procedure is described in detail below.

The invention further provides a method of determining whether a subject is suffering from melanoma. A DNA sample is obtained from a subject, and the level of DNA methylation in MINT17, MINT31, or the promoter region of GATA4 in the sample is determined. If the level of DNA methylation in MINT17, MINT31, or the promoter region of GATA4 in the sample is higher than that in normal cells, the subject is likely to be suffering from melanoma.

Similarly, the invention provides a method of determining whether a subject is suffering from breast cancer. A DNA sample is obtained from a subject, and the level of DNA methylation in MINT17 or MINT31 in the sample is determined. If the level of DNA methylation in MINT17 or MINT31 in the sample is higher than that in normal cells, the subject is likely to be suffering from breast cancer.

The level of DNA methylation may be represented by a methylation index of the methylated DNA copy number divided by the sum of the methylated DNA copy number and the unmethylated DNA copy number, the ratio of the methylated DNA copy number to the unmethylated DNA copy number, or the like.

“Normal cells” may be obtained from a normal subject or a normal tissue of a test subject. Preferably, the normal cells are obtained from a site where the cancer being tested for can originate or metastasize.

The invention also provides methods of determining melanoma or breast cancer status, monitoring cancer progression and treatment, and predicting the outcome of the cancer. These methods involve obtaining from a subject a sample containing melanoma or breast cancer cells and determining the level of DNA methylation at specific DNA locations in the cancer cells. Optionally, a method of the invention may include a step of comparing the levels of DNA methylation between samples obtained from different subjects, different sites on the same subject, or the same site on the same subject at different time points, for instance, at different cancer stages, or before, during, or after a cancer therapy (e.g., a surgery or chemotherapy).

The level of DNA methylation in MINT17, MINT31, or the promoter region of WIF1, TFPI2, RASSF1A, or SOCS1 is determining in melanoma cells. If the level of methylation in MINT17, MINT31, or the promoter region of WIF1, TFPI2, RASSF1A, or SOCS1 in the melanoma cells is higher than that in normal cells, the melanoma is likely to be an aggressive melanoma.

More specifically, if the level of DNA methylation in the promoter region of TFPI2 in the melanoma cells is higher than that in normal cells, the subject is suffering from AJCC (American Joint Committee on Cancer) Stage II, III, or IV melanoma. If the level of DNA methylation in the promoter region of RASSF1A in the melanoma cells is higher than that in normal cells, the subject is suffering from AJCC Stage III or IV melanoma.

Also, a higher level of DNA methylation in MINT31 or the promoter region of WIF1, TFPI2, RASSF1A, or SOCS1 in the melanoma cells indicates that the subject is suffering from a more aggressive melanoma. If the subject is suffering from an AJCC Stage I, II, or III melanoma, a higher level of DNA methylation in MINT17 in the melanoma cells indicates that the subject is suffering from a more aggressive melanoma. Similarly, if the subject is suffering from an AJCC Stage I, II, or IV melanoma, a higher level of DNA methylation in MINT17 in the melanoma cells indicates that the subject is suffering from a more aggressive melanoma.

The level of DNA methylation in MINT17, MINT31, or the promoter region of RASSF1A or RARβ2 is determining in breast cancer cells. If the level of DNA methylation in MINT17, MINT31, or the promoter region of RASSF1A or RARβ2 in the breast cancer cells is higher than that in normal cells, the breast cancer is likely to be an aggressive breast cancer.

More specifically, a higher level of DNA methylation in MINT17 or the promoter region of RASSF1A, or a lower level of DNA methylation in MINT31 in the breast cancer cells indicates that the breast cancer is more likely to be positive for ER (estrogen receptor); a higher level of DNA methylation in MINT17 or the promoter region of RASSF1A, or a lower level of DNA methylation in MINT31 or the promoter region of RARβ2 in the breast cancer cells indicates that the breast cancer is more likely to be positive for PR (progesterone receptor); a higher level of DNA methylation in the promoter region of RARβ2, or a lower level of DNA methylation in MINT17 in the breast cancer cells indicates that the breast cancer is more likely to be positive for HER2 (human epidermal growth factor receptor 2); and a higher level of DNA methylation in MINT31 in the breast cancer cells indicates that the subject is likely to be suffering from a more aggressive breast cancer.

As used herein, by a breast cancer that is “positive” for ER, PR, or HER2 is meant that ER, PR, or HER2 is expressed in the breast cancer.

To predict the outcome of melanoma, the level of DNA methylation in MINT31 is determining in the melanoma cells. A higher level of methylation in MINT31 in the melanoma cells indicates a more likelihood of disease-free survival and overall survival.

To predict the outcome of breast cancer, the level of DNA methylation in MINT17, MINT31, or the promoter region of RARβ2 is determining in the breast cancer cells. A higher level of methylation in MINT17, MINT31, or the promoter region of RARβ2 in the breast cancer cells indicates a less likelihood of overall survival.

The discovery of DNA methylation in MINT17, MINT31, or the promoter region of GATA4 in melanoma cells and DNA methylation in MINT17 or MINT31 in breast cancer cells is useful for identifying candidate compounds for treating melanoma and breast cancer. Briefly, a melanoma or breast cancer cell is contacted with a test compound. The levels of DNA methylation in MINT17, MINT31, or the promoter region of GATA4 in the melanoma cell or DNA methylation in MINT17 or MINT31 in the breast cancer cell prior to and after the contacting step are compared. If the level of the DNA methylation in the cell decreases after the contacting step, the test compound is identified as a candidate for treating melanoma or breast cancer.

The test compounds can be obtained using any of the numerous approaches (e.g., combinatorial library methods) known in the art. See, e.g., U.S. Pat. No. 6,462,187. Such libraries include, without limitation, peptide libraries, peptoid libraries (libraries of molecules having the functionalities of peptides, but with a novel, non-peptide backbone that is resistant to enzymatic degradation), spatially addressable parallel solid phase or solution phase libraries, synthetic libraries obtained by deconvolution or affinity chromatography selection, and the “one-bead one-compound” libraries. Compounds in the last three libraries can be peptides, non-peptide oligomers, or small molecules. Examples of methods for synthesizing molecular libraries can be found in the art. Libraries of compounds may be presented in solution, or on beads, chips, bacteria, spores, plasmids, or phages.

The compounds so identified are within the invention. These compounds and other compounds known to inhibit DNA methylation or promote demethylation of DNA can be used for treating melanoma or breast cancer by administering an effective amount of such a compound to a subject suffering from melanoma or breast cancer.

A subject to be treated may be identified in the judgment of the subject or a health care professional, and can be subjective (e.g., opinion) or objective (e.g., measurable by a test or diagnostic method such as those described above).

A “treatment” is defined as administration of a substance to a subject with the purpose to cure, alleviate, relieve, remedy, prevent, or ameliorate a disorder, symptoms of the disorder, a disease state secondary to the disorder, or predisposition toward the disorder.

An “effective amount” is an amount of a compound that is capable of producing a medically desirable result in a treated subject. The medically desirable result may be objective (i.e., measurable by some test or marker) or subjective (i.e., subject gives an indication of or feels an effect).

For treatment of cancer, a compound is preferably delivered directly to tumor cells, e.g., to a tumor or a tumor bed following surgical excision of the tumor, in order to treat any remaining tumor cells.

The identified compounds can be incorporated into pharmaceutical compositions. Such compositions typically include the compounds and pharmaceutically acceptable carriers. “Pharmaceutically acceptable carriers” include solvents, dispersion media, coatings, antibacterial and antifungal agents, isotonic and absorption delaying agents, and the like, compatible with pharmaceutical administration.

A pharmaceutical composition is formulated to be compatible with its intended route of administration. See, e.g., U.S. Pat. No. 6,756,196. Examples of routes of administration include parenteral, e.g., intravenous, intradermal, subcutaneous, oral (e.g., inhalation), transdermal (topical), transmucosal, and rectal administration.

It is advantageous to formulate oral or parenteral compositions in dosage unit form for ease of administration and uniformity of dosage. “Dosage unit form,” as used herein, refers to physically discrete units suited as unitary dosages for the subject to be treated, each unit containing a predetermined quantity of an active compound calculated to produce the desired therapeutic effect in association with the required pharmaceutical carrier.

The dosage required for treating a subject depends on the choice of the route of administration, the nature of the formulation, the nature of the subject's illness, the subject's size, weight, surface area, age, and sex, other drugs being administered, and the judgment of the attending physician. Suitable dosages are in the range of 0.01-100.0 mg/kg. Wide variations in the needed dosage are to be expected in view of the variety of compounds available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the compound in a suitable delivery vehicle (e.g., polymeric microparticles or implantable devices) may increase the efficiency of delivery, particularly for oral delivery.

The melanoma or breast cancer may be a primary cancer, metastatic cancer, or aggressive cancer. As used herein, an “aggressive cancer” refers to a caner that invades, metastasizes to distant organ sites, and grows fast, or a cancer that is capable of invading, metastasizing to distant organ sites, and growing fast. Aggressive cancers include cancers at various AJCC stages, e.g., AJCC Stage I, II, III, or IV, or cancers at more advanced AJCC stages.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLES

### Example I

### CpG Island Methylator Phenotype Predicts Progression of Malignant Melanoma

### Abstract

Purpose: The CpG island methylator phenotype (CIMP) may be associated with development of malignancy through coordinated inactivation of tumor-suppressor and tumor-related genes (TRGs) and methylation of multiple noncoding, methylated-in-tumor (MINT) loci. These epigenetic changes create a distinct CIMP pattern that has been linked to recurrence and survival in gastrointestinal cancers. Because epigenetic inactivation of TRGs also has been shown in malignant melanoma, we believed the existence of a clinically significant CIMP in cutaneous melanoma progression. Experimental Design: The methylation status of the CpG island promoter region of TRGs related to melanoma pathophysiology (WIF1, TFPI2, RASSF1A, RARβ2, SOCS1, and GATA4) and a panel of MINT loci (MINT1, 2, 3, 12, 17, 25, and 31) in primary and metastatic tumors of different clinical stages (n=122) was assessed. Results: Here, we show an increase in hypermethylation of the TRGs WIF1, TFPI2, RASSF1A, and SOCS1 with advancing clinical tumor stage. Furthermore, we find a significant positive association between the methylation status of MINT17, MINT31, and TRGs. The methylation status of MINT31 is associated with disease outcome in stage III melanoma. Conclusions: These findings demonstrate the significance of a CIMP pattern that is associated with advancing clinical stage of malignant melanoma.

### Materials and Methods

### Cell Lines

HeMnMP, a moderately pigmented human melanocyte strain, was obtained from Cascade Biologics and maintained in Medium 254 with human melanocyte growth supplement (Portland, Oreg.). A dermal fibroblast cell line originating from a healthy donor was established and kindly donated by the Osaka University Department of Dermatology (Osaka, Japan), and was maintained in Dulbecco's modified Eagle's medium supplemented with 10% heat-inactivated fetal calf serum. Twelve melanoma cell lines were established from metastatic tumors at the John Wayne Cancer Institute (JWCI) and maintained in RPMI-1640 supplemented with penicillin, streptomycin, and 10% heat-inactivated fetal calf serum. All cultures were maintained at 37° C., 5% CO₂ in a humidified incubator.

### Clinical Specimens

Approval for the use of human tissues was obtained from the JWCI/Saint John's Health Center (SJHC) Institutional Review Board before study initiation. Analysis was undertaken of 122 paraffin-embedded archival tissue (PEAT) specimens from 107 patients diagnosed with malignant melanoma by the Division of Surgical Pathology at SJHC. Specimens were classified using the 2002 American Joint Committee on Cancer (AJCC) staging criteria for cutaneous melanoma (20). Of the 122 PEAT melanoma specimens, 35 were from primary tumors associated with AJCC stage I (n=18) and stage II (n=17) disease. A total of 25 stage III patients were included. Of these, 7 had only primary tumor specimens available for analysis, and 8 had only specimens from nodal metastases. For 10 patients, both primary and nodal specimens were available.

Normal skin control samples were obtained from tumor-free areas of primary melanoma tissue blocks. Clinical characteristics of the enrolled patients are summarized in Table 1. The patients consisted of 39 females and 68 males between 12 and 88 years of age. Breslow thickness data were available for 48 of 52 patients with primary tumor specimens, and 41 of 55 patients with regional lymph node or distant organ metastases. Mean clinical follow-up was 38.9 months (range 0 to 328).

TABLE 1
Clinical characteristics of melanoma patients and tissue samples

| Characteristics | N (%) |
|---|---|
| Total patients | 107 |
| Age | |
| Mean ± SD | 59.4 ± 16.64 |
| Median, min-max | 60, 12-88 |
| <50 | 27 (25.2) |
| ≧50 | 80 (74.8) |
| Gender | |
| F | 39 (36.4) |
| M | 68 (63.6) |
| Breslow thickness | |
| ≦1.0 | 19 (17.8) |
| 1.01-2.0 | 22 (20.6) |
| 2.01-4.0 | 32 (29.9) |
| >4.0 | 16 (15) |
| Unknown | 18 (16.8) |
| Total tissue samples | 122 |
| AJCC stage | |
| I | 18 (14.8) |
| II | 17 (13.9) |
| III (primary tumor only) | 7 (5.7) |
| III (lymph node metastasis only) | 8 (6.6) |
| III (primary tumor and lymph node metastasis) | 10 (8.2) |
| IV (metastasis) | 52 (42.6) |
| skin/soft tissue | 12 (23.1) |
| Lung | 11 (21.2) |
| adrenal gland | 10 (19.2) |
| lymph node | 8 (15.4) |
| small bowel | 6 (11.5) |
| Other | 5 (9.6) |

Paired early and advanced-stage specimens were available for 16 patients. Thirteen patients had primary tumor specimens with subsequent nodal (n=10) or distant (n=3) metastatic tumor specimens, and 3 patients had nodal metastases followed by distant metastases. Of the 18 stage III patients with specimens from lymph node metastases, 10 had primary tumor specimens available, and 3 subsequently developed distant metastatic lesions. These paired tumor specimens were used to examine differences in methylation with stage progression on a per-patient basis.

Sites of distant metastasis for the 52 stage IV patients studied included skin or subcutaneous tissue (n=12), lung (n=11), adrenal gland (n=10), non-regional lymph nodes (n=8), small bowel (n=6), and others (n=5).

### DNA Isolation

Sections of 8 μm were cut from formalin-fixed, PEAT blocks. An H&E slide was prepared for each sample to confirm tumor location and to assess tissue homogeneity by light microscopy. Tumor tissues were isolated using manual microdissection. To extract DNA, dissected tissues were digested with 100 μL of lysis buffer containing 2.4 mAU Proteinase K (Qiagen, Valencia, Calif.) at 50° C. overnight, followed by heat inactivation of proteinase K at 95° C. for 15 mM. DNA was purified with phenol-chloroform-isoamyl alcohol (Fisher Scientific, Pittsburgh, Pa.), precipitated by ethanol, and quantified using the PicoGreen Assay (Molecular Probes, Invitrogen, Carlsbad, Calif.). DNA from cell lines was isolated using DNAzol Genomic DNA Isolation Reagent (Molecular Research Center, Inc., Cincinnati, Ohio) according to the manufacturer's recommendations, then quantified and assessed for purity by UV spectrophotometry. Extracted DNA was subjected to sodium bisulfite modification (SBM) as described previously (6).

### Epigenetic Changes Detected by Methylation-Specific PCR (MSP)

Methylation status was assessed for each gene using two sets of fluorescent-labeled primers designed to amplify methylated or unmethylated DNA sequences. Methylated and unmethylated primer sequences are summarized in Supplemental Appendix Table 1a. Primers were designed using MethPrimer (21). Bisulfite-modified DNA was subjected to PCR amplification in a final reaction volume of 10 μl containing PCR buffer, 2.5-4.5 mM MgCl₂, 0.8 mM dNTPs, 0.3 μM primers, and 0.5 U of AmpliTaq Gold DNA polymerase (Applied Biosystems, Foster City, Calif.). PCR amplification was performed with an initial 10-min incubation at 95° C., followed by 36-40 cycles of denaturation at 95° C. for 30 sec, annealing for 30 sec, extension at 72° C. for 45 sec, and a final 7-min hold at 72° C. Lymphocyte DNA obtained from healthy donors and amplified by phi-29 DNA polymerase served as a positive unmethylated control after SBM (22). SssI methylase (New England Bio Labs, Beverly, Mass.)-treated lymphocyte DNA served as a positive methylated control. Unmodified lymphocyte DNA was used as a negative control for methylated and unmethylated reactions.

SUPPLEMENTAL APPENDIX TABLE 1a
MSP primers used for tumor-related genes

| | | | Annealing | PCR |
|---|---|---|---|---|
| | | | Temper- | product |
| | | Antisense primer | ature | size (base |
| Gene | Sense primer (5′- to -3′) | (5′- to -3′) | (° C.) | pairs) |
| WIF-1 | | | | |
| M | GGGCGTTTTATTGGGCGTAT | AACCTAAACGACCGCCACTT | 63 | 116 |
| U | GGGTGTTTTATTGGGTGTAT | AACCTAAACAACCACCACTTA | 58 | 116 |
| TFPI-2 | | | | |
| M | TTTCGTATAAAGCGGGTATTC | ACGACCCGCTAAACAAAACG | 60 | 95 |
| U | GGATGTTTGTTTTGTATAAAGTG | AAACATCCAAAAAAACACCTAAC | 60 | 89 |
| RASSF1A | | | | |
| M | GTGTTAACGCGTTGCGTATC | AACCCCGCGAACTAAAAACGA | 60 | 93 |
| U | TTTGGTTGGAGTGTGTTAATG | CAAACCCCACAAACTAAAAACAA | 60 | 105 |
| RARβ2 | | | | |
| M | GAACGCGAGCGATTCGAGT | GACCAATCCAACCGAAACG | 59 | 142 |
| U | GGATTGGGATGTTGAGAATGT | CAACCAATCCAACCAAAACAA | 59 | 158 |
| SOCS-1 | | | | |
| M | TCGTTCGTCGTCGATTATC | AAAAAAATACCCACGAACTCG | 61 | 132 |
| U | TATTTTGTTTGTATGTTGATTATTG | AAACTCAACACACAACCACTC | 57 | 122 |
| GATA 4 | | | | |
| M | TATAGCGAATTTAATCGATTTTCG | GACTACACCTCCGCTAAACG | 60 | 164 |
| U | TGGGTATTATAGTGAATTTAATTGATTTTT | CCCAACTACACCTCCACTAAACA | 60 | 175 |

M: Methylated;
U: Unmethylated

### Capillary Array Electrophoresis (CAE)

PCR products were assessed using CAE (CEQ 8000XL; Beckman Coulter, Inc., Fullerton, Calif.) as previously described (6) using Beckman Coulter WellRED dye-labeled phosphoramidites (Genset Oligos, La Jolla, Calif.). Forward methylated sequence-specific primers were labeled with D4 dye, and forward unmethylated sequence-specific primers were labeled with D3 dye. One μL of methylated PCR product and one μL of unmethylated PCR product were mixed with loading buffer and a dye-labeled size standard (Beckman Coulter) and loaded in a 96-well plate for CEQ peak ratio analysis. Samples demonstrating only a peak for D3 dye (representing unmethylated DNA) were marked as unmethylated. Samples demonstrating a peak for D4 dye (representing methylated DNA), or peaks for both methylated and unmethylated DNA, were marked as methylated.

### Absolute Quantitative Analysis of Methylated Alleles (AQAMA)

To quantify the methylation status of seven MINT loci (MINT1, 2, 3, 12, 17, 25, and 31), we employed the AQAMA assay as previously described (3). A single set of PCR primers was designed to amplify bisulfate-modified DNA for both methylated and unmethylated sequences, using Primer 3 software (see the website at frodo.wi.mit.edu/cgi-bin/primer3/primer3_www.cgi). The methylation status of CpG's was distinguished by two different minor groove binder (MGB)-molecule-containing probes (Applied Biosystems), specific for either methylated or unmethylated sequences, designed with Primer Express software (version 2.0, Applied Biosystems). Methylated and unmethylated probes were labeled with FAM (6-carboxyfluorescein) and VIC™, respectively. Black hole quenchers (BHQ) were used to silence the probes' fluorescent signals when not hybridized. The sequences of primer sets and MGB probes are listed in Supplemental Appendix Table 1b.

SUPPLEMENTAL APPENDIX TABLE 1b
AQAMA conditions
used for MINT Loci

| | MINT17 | MINT31 |
|---|---|---|
| Primer set | | |
| (5′- to -3′) | | |
| Sense | AGGGGTTAGGT | TAAAGTGAGG |
| | TGAGGTTGTT | GGTGGTGATG |
| Antisense | TCTACCTCTTC | AAAAACACTTC |
| | CCAAATTCCA | CCCAACATCT |
| MGB probes | | |
| M | FAM-TTGGAT | FAM-AGGTTT |
| | GGATCGCGG | CGTCGTGTTT |
| U | VIC-TATTTTGGA | VIC-AGGTTTTG |
| | TGGATTGTGG | TTGTGTTTAT |

M: Methylated;
U: Unmethylated

Real-time PCR for the AQAMA assay was performed as described previously (3). The reaction mixture totaling 10 μl for each AQAMA PCR consisted of 1 μl modified template DNA, PCR buffer, 0.4 μM of each forward and reverse primer, 1.4 U of iTaq DNA polymerase (Bio-Rad Laboratories, Hercules, Calif.), 0.6 mM of dNTPs, 0.025 pM of each MGB probe, and 4.5 mM of MgCl₂. The mixture was processed by a 2-step PCR method using ABI Prism 7900HT Sequence Detection System (Applied Biosystems) with an initial heating at 95° C. for 10 min, followed by 40 cycles of denaturation at 95° C. for 15 sec, and annealing and extension at 60° C. (58° C. for MINT3 and 25) for 60 sec. The obtained PCR amplification curves from methylated and unmethylated alleles were analyzed with SDS software version 2.3 (Applied Biosystems). The final data output was reported as “methylation index” (MI=methylated copy number/[methylated copy number+unmethylated copy number]). All experiments were performed in duplicate; mean values from duplicate measurements were used for calculation of the MI. Control DNA from methylated lymphoblastoid cell lines (AGS and Raji) or unmethylated gastric cancer cell lines (RL-0380 and FN-0028 from JWCI) was used to verify the reproducibility and accuracy of this assay. To quantify methylated and unmethylated copy numbers, a standard curve was created using high fidelity and quality-constructed plasmids for methylated and unmethylated sequences as previously described (3). The mean methylation index plus one standard deviation obtained from 12 non-tumor skin specimens was used as the cut-off point to separate the methylated and unmethylated samples.

### Statistical Analysis

Categorical data was analyzed using the chi-square test; Fisher's exact test was used in the case of small sample sizes. Two-tailed p-values of <0.05 were considered statistically significant. Bonferroni correction was applied to multiple comparisons. Trend analysis of methylation across AJCC stages was performed using the Cochran Armitage test. McNemar's test was used to compare the methylation frequency of different stage samples obtained from the same patient. Cox's proportional hazard regression models were created for OS and DFS calculations incorporating multiple variables. The factors for multivariate analysis included presence or absence of methylation for each marker, gender, age, Breslow thickness, and presence or absence of ulceration, each as independent variables. Survival curves were constructed using the log-rank method. All statistical calculations were performed using SAS software version 8.02 (SAS Institute Inc.).

### Results

### Methylation Profiles of Cell Lines

MSP and AQAMA primers and probes were initially screened using eight melanoma tumor specimens to detect promoter methylation of six TRGs and methylation of seven MINT loci, respectively. Two of the seven MINT loci from these initial screening analyses demonstrated a significant difference in methylation frequency in the tumor specimens as compared with tumor-free skin portions of the same patient samples. Other MINT loci in the initial screening analysis showed similar high frequencies of methylation (MINT12) or low to absent methylation (MINT1, 2, 3, 25) in both tumors and normal skin. Therefore, further analyses by AQAMA focused on MINT17 and MINT31. Initial screening analysis of the six TRGs similarly demonstrated high frequencies of promoter methylation in the eight tumor tissues tested as compared to uniform absence of promoter methylation in the tumor-free skin portions of the same patient samples.

WIF1, TFPI2, RASSF1A, RARβ2, SOCS1, GATA4, MINT17, and MINT31 were each methylated in at least 50% of the 12 melanoma cell lines tested (Table 2). All biomarkers were methylated in cell lines M1 to M3, whereas none were methylated in M11 and M12. All biomarkers were unmethylated in melanocyte and dermal fibroblast cell lines.

TABLE 2
Promoter hypermethylation of TRGs and MINT loci
in melanoma cell lines

| | | | | | | | | | Markers |
|---|---|---|---|---|---|---|---|---|---|
| Cell Lines | WIF1 | TFPI2 | RASSF1A | RARβ2 | SOCS1 | GATA4 | MINT17 | MINT31 | Methylated |
| M1 | M | M | M | M | M | M | M | M | 8 |
| M2 | M | M | M | M | M | M | M | M | 8 |
| M3 | M | M | M | M | M | M | M | M | 8 |
| M4 | M | M | M | M | M | U | M | M | 7 |
| M5 | M | M | M | M | M | M | U | M | 7 |
| M6 | U | M | M | M | M | U | M | M | 6 |
| M7 | M | M | U | M | U | M | M | M | 6 |
| M8 | U | U | M | M | M | U | M | M | 5 |
| M9 | M | U | U | M | M | M | U | M | 5 |
| M10 | U | U | U | U | U | U | U | M | 1 |
| M11 | U | U | U | U | U | U | U | U | 0 |
| M12 | U | U | U | U | U | U | U | U | 0 |
| Melanocyte | U | U | U | U | U | U | U | U | 0 |
| Dermal | U | U | U | U | U | U | U | U | 0 |
| fibroblast | | | | | | | | | |
| Methylated | 58.3 | 58.3 | 58.3 | 75 | 66.7 | 50 | 58.3 | 83.3 | |
| Lines (%) | | | | | | | | | |

M: Methylated;
U: Unmethylated

### Methylation Profiles in Melanomas

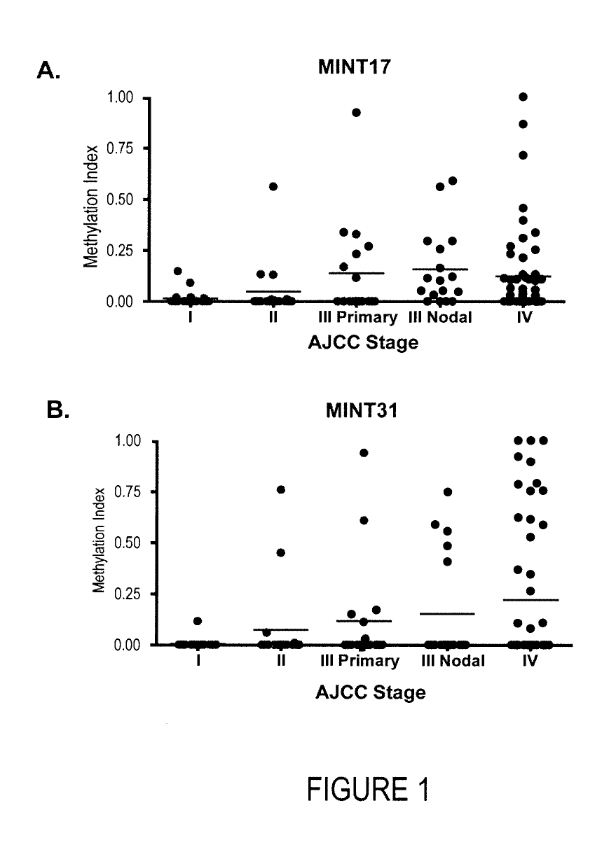

The MI obtained for MINT17 and 31 loci, stratified by AJCC stage, are depicted in FIGS. 1A and 1B, respectively. Overall methylation percentages stratified by AJCC stage for each of the six TRGs and the two MINT loci are reported in Table 3a. Univariate analysis revealed no significant difference in methylation status by age or gender.

TABLE 3a
Percent of melanoma tissues
exhibiting methylation of TRGs and MINT loci

| AJCC | | | | | | | | | |
|---|---|---|---|---|---|---|---|---|---|
| Stage | ( n ) | MINT17 | MINT31 | WIF1 | TFPI2 | RASSF1A | RARβ2 | SOCS1 | GATA4 |
| I (P) | ( 18 ) | 11.1 | 5.6 | 5.6 | 0 | 0 | 58.3 | 7.1 | 16.7 |
| II (P) | ( 17 ) | 17.6 | 23.5 | 6.3 | 6.3 | 0 | 66.7 | 23.1 | 8.3 |
| III (P) | ( 17 ) | 41.2 | 35.3 | 31.3 | 17.6 | 26.7 | 64.3 | 25 | 42.9 |
| III (M) | ( 18 ) | 52.9 | 27.8 | 50 | 17.6 | 27.8 | 47.1 | 23.5 | 22.2 |
| IV (M) | ( 52 ) | 38.5 | 36.5 | 44 | 44.9 | 48.9 | 56.3 | 44.9 | 34 |
| Overall | ( 122 ) | 34.2 | 28.1 | 33.3 | 21.7 | 28.6 | 57.6 | 31.5 | 27.5 |

(P) Primary;
(M) Metastatic.
For additional information on specimen, see Table 1.
Bold type: refers to ≧50%.

Advancing AJCC stage was associated with increased methylation of MINT17 (P=0.0004), MINT31 (P=0.026), TFPI2 (P=0.001), WIF1 (P=0.002), SOCS1 (P=0.009), and RASSF1A (P<0.0001), but not GATA4 and RARβ2, as determined by the Cochran Armitage test. This finding was most pronounced for TFPI2 and RASSF1A, which were uniformly unmethylated (0%) in stage I primary tumor specimens, whereas the methylation frequency of these genes was 45% and 49% in stage IV metastatic specimens, respectively. Conversely, RARβ2 was found to be highly methylated in early stage primary tumors (58% and 67% for stage I and II specimens, respectively). Similarly, 17% of stage I primary tumors demonstrated GATA4 methylation, which did not reliably or significantly increase with advancing stage. Significant increases in the methylation frequencies of MINT17, MINT31, and the TRGs WIF1, TFPI2, RASSF1A, and SOCS1 were found when comparing stage I primary tumors vs. stage IV metastatic tumors, but not stage I vs. stage II, or stage III vs. stage IV (Table 3b). For MINT17 and WIF1, a decrease in the percentage of hypermethylated specimens was noted from stage III (nodal) to stage IV. There were no significant differences observed in methylation frequency or MI between different anatomic sites of distant metastasis. Both early and advanced-stage paired tumors were available for 16 patients in our study group. Examination of these paired tumors demonstrated a significant increase of WIF1 methylation with AJCC stage progression (P=0.01).

TABLE 3b
Difference in methylation status between AJCC stages

| Stage Comparison | MINT17 | MINT31 | WIF1 | TFPI2 | RASSF1A | RARβ2 | SOCS1 | GATA4 |
|---|---|---|---|---|---|---|---|---|
| stage I vs. II | NS | NS | NS | NS | NS | NS | NS | NS |
| stage I vs. III | 0.049 | 0.036 | NS | NS | NS | NS | NS | NS |
| Stage I vs. IV* | 0.021 | 0.005 | 0.003 | 0.006 | <.0001 | NS | 0.005 | NS |
| Stage II vs. III | NS | NS | NS | NS | 0.015 | NS | NS | 0.039 |
| Stage II vs. IV* | NS | NS | 0.015 | 0.013 | <.0001 | NS | NS | 0.055 |
| stage III vs. IV* | NS | NS | NS | NS | NS | NS | NS | NS |

P value analyzed by Fisher's exact test;
NS: Not Significant
*For comparisons with stage IV, Chi-square test was used.
P values < 0.0083 are significant after Boniferroni correction shown in bold type).

### Relationship Between Methylation Status of MINT-Loci and Tumor-Related Genes

Positive relationships were found for methylation of MINT17 with MINT31, TFPI2, WIF1, and SOCS1 (Table 4). MINT31 methylation was positively associated with methylation of all six TRGs. Methylation of TFPI2 and WIF1 was also associated with methylation of the other TRGs. There was no statistically significant relationship between methylation of MINT17 as compared with GATA4, RASSF1A, or RARβ2; methylation of GATA4 was associated with RASSF1A and RARβ2 methylation, however. The absence of a methylation relationship was also noted for SOCS1 as compared with GATA4 and RARβ2, as well as RASSF1A with RARβ2.

TABLE 4
Relationship between methylation status of MINT loci and TRGs

| Biomarker | Comparison | P value* |
|---|---|---|
| MINT17 | MINT31 | 0.033 |
| | TFPI2 | 0.014 |
| | WIF1 | 0.002 |
| | SOCS1 | 0.013 |
| | GATA4 | NS |
| | RASSF1A | NS |
| | RARβ2 | NS |
| MINT31 | TFPI2 | 0.002 |
| | WIF1 | 0.009 |
| | SOCS1 | 0.002 |
| | GATA 4 | <0.0001 |
| | RASSF1A | 0.002 |
| | RARβ2 | 0.042 |
| TFPI2 | WIF1 | <0.0001 |
| | SOCS1 | 0.0003 |
| | GATA4 | 0.0001 |
| | RASSF1A | <0.0001 |
| | RARβ2 | 0.008 |
| WIF1 | SOCS1 | 0.001 |
| | GATA4 | 0.002 |
| | RARβ2 | 0.005 |
| SOCS1 | GATA4 | NS |
| | RASSF1A | 0.023 |
| | RARβ2 | NS |
| GATA4 | RASSF1A | <0.0001 |
| | RARβ2 | 0.005 |
| RASSF1A | RARβ2 | NS |

*2-tailed Chi Square Test. All significant relationships are positive. NS = Not significant.

### MINT31 Hypermethylation Predicts Improved Disease-Free and Overall Survival

Disease free and overall survival rates in stage I and II malignant melanoma are very high. Conversely, stage IV disease is marked by a much shorter median survival that is further impacted by site of metastasis. Therefore, the assessment of disease outcome in relation to methylation of specific genes and loci was limited to AJCC stage III patients only. Survival analysis was conducted for all stage III patients stratified by biomarker methylation status (n=25). Stage III patients with primary versus nodal metastatic specimens were compared in a univariate analysis for differences in biomarker methylation, Breslow depth, Clark level, gender, histological type, and tumor ulceration; no statistically significant differences were found. Of the 25 AJCC stage III patients analyzed, clinical treatment consisted of multimodal therapy including surgery, vaccine therapy, chemotherapy, non-specific or intratumoral Bacillus Calmette-Guerin (BCG), cytokine therapy (IL-2 and/or interferon) and radiation. After confirming the absence of statistically significant differences in clinicopathologic features and biomarker methylation within the AJCC stage III patient population, a multivariate survival analysis was performed.

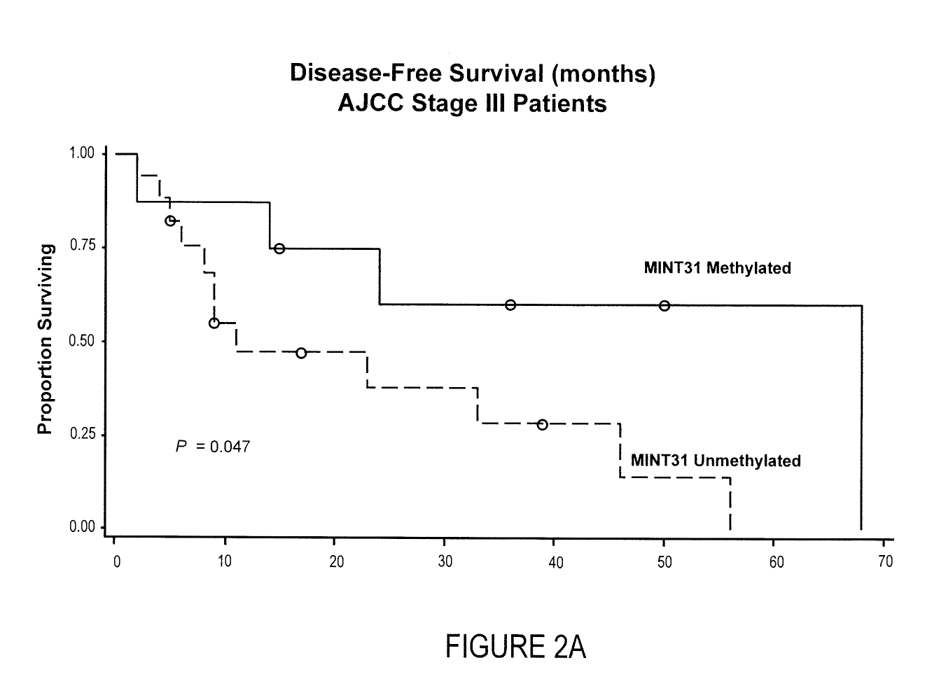

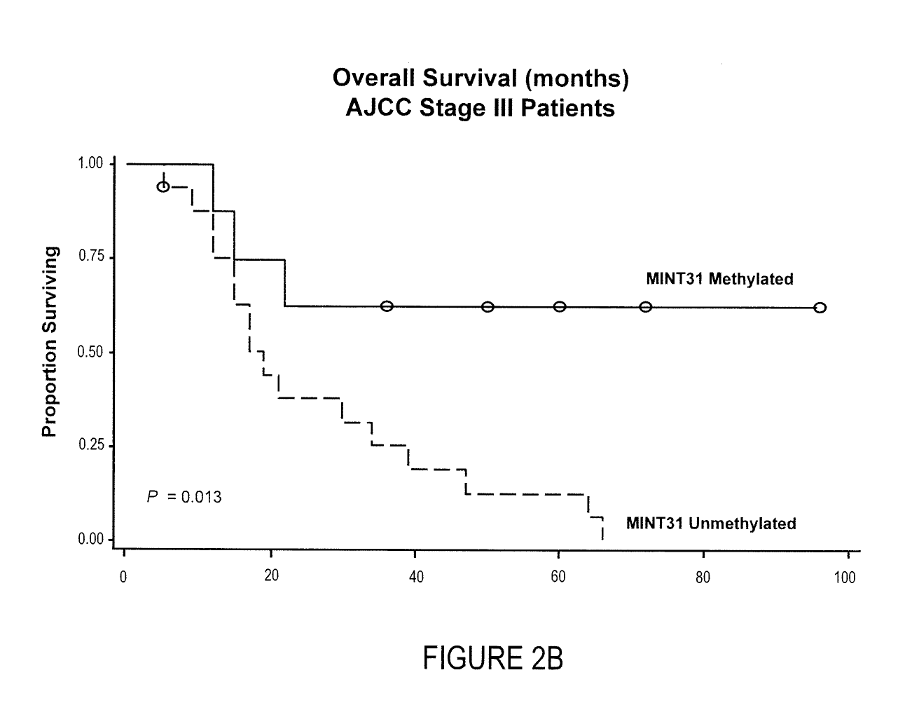

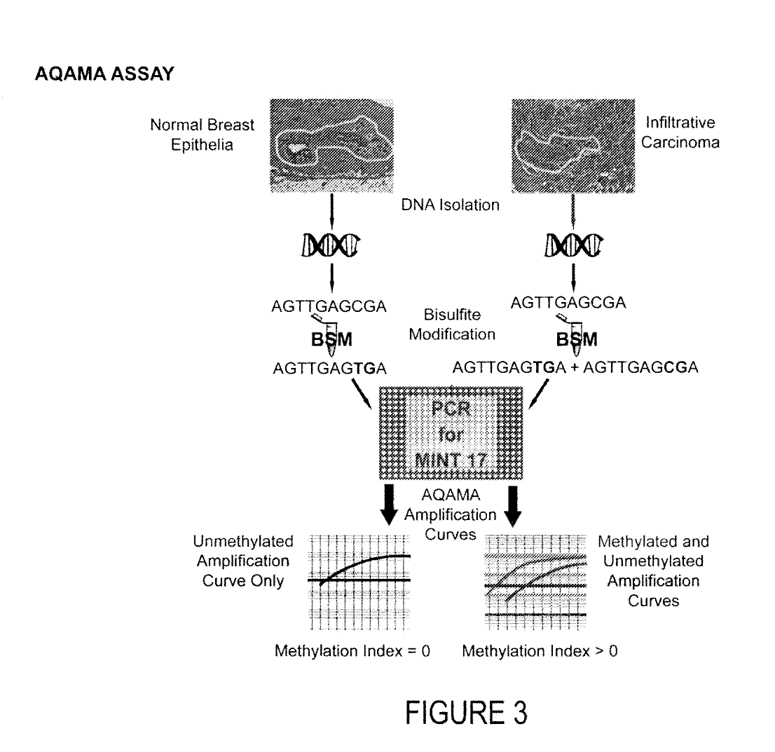

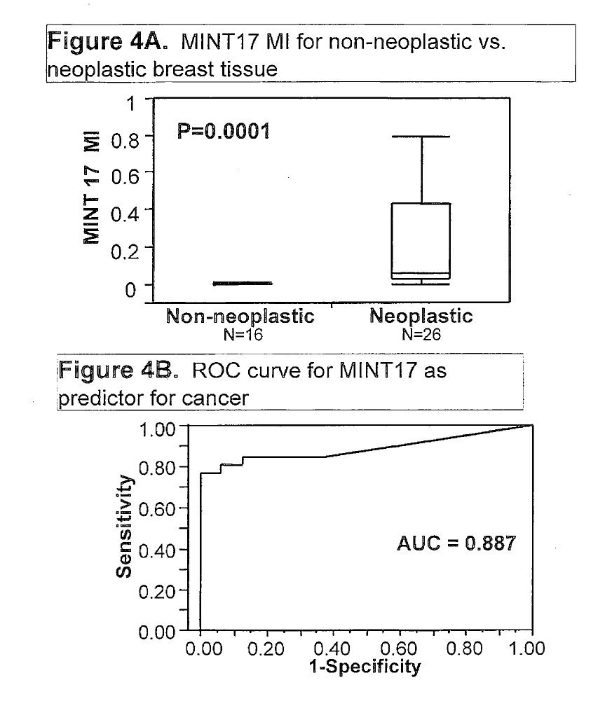

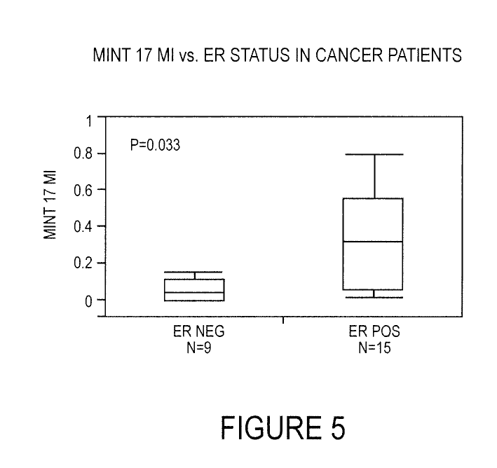

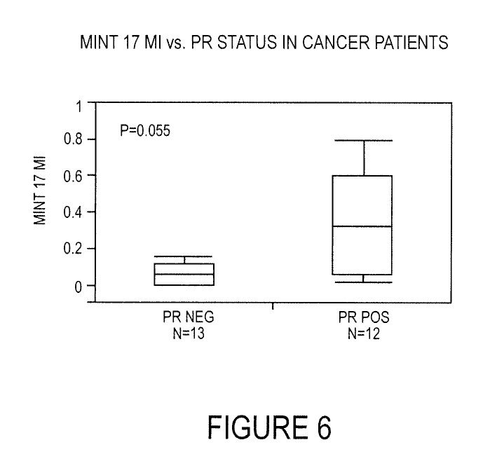

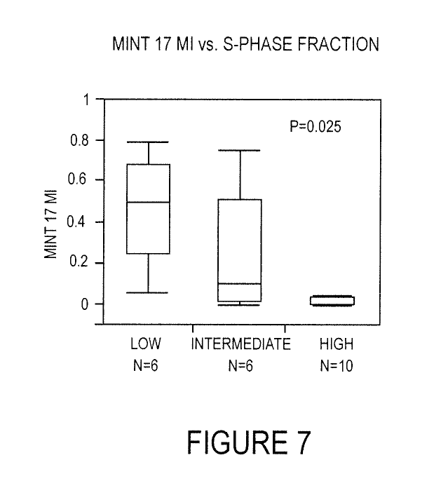

MINT31 methylation was found to be a significant predictor of improved overall survival (Cox's proportional hazard regression model, HR=0.237; P=0.024) for all 25 patients in our study with AJCC stage III disease. The log-rank test confirmed both disease-free and overall survival benefits with MINT31 methylation (FIGS. 2A and B, P=0.047 and 0.013, respectively). No adverse or beneficial effects on clinical outcome were noted with methylation of any of the other biomarkers tested.

### Discussion

Our study investigated the clinical significance of CpG island methylation status in the evolution and progression of malignant melanoma. Analysis of primary and metastatic tumors across different clinical stage groupings provided a unique opportunity to determine whether these epigenetic changes are related to tumor progression. Aberrant hypermethylation of the genomic markers was not present in normal melanocytes or fibroblasts, but was identified to varying degrees in primary and metastatic tumor tissues. Methylation of MINT17, MINT31, TFPI2, WIF1, RASSF1A, and SOCS1 increased significantly with advancing clinical stage, strongly suggesting that inactivation of these genes and loci is associated with tumor progression. These findings in melanoma are consistent with previous reports of MINT methylation as a determinant of a cancer-specific CIMP in gastric and colorectal cancers (4, 18), and of the association of TRG hypermethylation with melanoma and other cancers (6, 9, 10, 15).

For MINT17 and WIF1 in particular, it was interesting to note that lower methylation percentages were found for stage IV (distant) metastatic specimens in contrast with stage III (nodal) metastases. One plausible explanation could be that hypermethylation of MINT17 and WIF1 is involved with the initiation of the metastatic process, such that tumor clones with a higher degree of hypermethylation are more likely to migrate to and establish metastases in regional lymph nodes, whereas those tumor cells with a lower degree of hypermethylation are more suited to formation of distant metastases. Alternatively, the tumor microenvironment may select for the establishment of specific tumor cell clones expressing particular methylation patterns.

Paired analyses of patients with tumor specimens from both early and advanced-stage of disease showed significant increases in WIF1 methylation with melanoma stage progression. To our knowledge, this is the first clinical evidence of the role of WIF1 methylation in melanoma progression. These data strongly support the results of earlier in nitro and animal studies of the involvement of Wnt signaling in melanoma tumor growth, the ability to inhibit tumor growth with the restoration of WIF1 expression, and the potential use of Wnt pathway inhibition as a targeted therapy for high-risk or metastatic melanoma (15).

In contrast, RARβ2 methylation was seen in 58% of all tumor specimens tested without a detectable association with AJCC stage, implying that epigenetic inactivation of this particular gene may be a very early event in tumorigenesis. Although there was considerable variability in GATA4 methylation status across tumor stage groupings, GATA4 methylation was identified in a significant percentage of stage I tumors (Table 3a) but not in the melanocyte or dermal fibroblast cell lines (Table 2) or normal skin specimens. This implies that GATA4 activation may play a role in tumor suppression, which is consistent with previous reports of its function in other cancers (16).

Of particular interest in this study were the positive relationships between the methylation status of MINT loci and TRGs because a methylator phenotype based on multiple TRGs may have more prognostic clinical value than the methylation status of any one particular TRG. Methylation of MINT31 was positively associated with methylation of all six TRGs, as was methylation of TFPI2 and WIF1. While methylation of MINT17 was associated with methylation of MINT31, TFPI2, WIF1, and SOCS1, no relation was found between MINT17 methylation and GATA4, RASSF1A, or RARβ2. A pattern emerging from these data suggests that MINT17 methylation is a particularly sensitive marker for disease progression because it is present in conjunction with methylation of the TRGs that are strongly associated with advancing clinical stage. Because MINT31 methylation is associated with methylation of all of the TRGs, it is perhaps more suitable as a biomarker of disease presence or absence. MINT17 and MINT31 methylation may therefore be representative of a CIMP for malignant melanoma. Potential clinical applications of this knowledge include the testing of primary melanomas for MINT17 hypermethylation and, used in conjunction with clinicopathologic factors such as Breslow depth, Clark level, ulceration and mitotic rate, to offer further treatment such as lymph node biopsy based on the result.

This study included a preliminary analysis of survival in a subgroup of patients with stage III melanoma. Survival plots stratified by methylation status were notable for improved disease-free and overall survival associated with methylation of MINT31 but not of any other biomarkers. It is conceivable that alterations in the activation status of additional genes or gene products other than those examined here may result in phenotypic changes leading to slower disease progression and/or tumor cell doubling times, or perhaps improved recognition of tumor cells by the immune system.

### REFERENCES

1. Jemal A, Siegel R, Ward E, Murray T, Xu J, Thun M J. Cancer statistics, 2007. CA Cancer J Clin 2007; 57:43-66.

2. Herman J G, Baylin S B. Gene silencing in cancer in association with promoter hypermethylation. N Engl J Med 2003; 349:2042-54.

3. de Maat M F, Umetani N, Sunami E, Turner R R, Hoon D S. Assessment of methylation events during colorectal tumor progression by absolute quantitative analysis of methylated alleles. Mol Cancer Res 2007; 5:461-71.

4. Kusano M, Toyota M, Suzuki H, et al. Genetic, epigenetic, and clinicopathologic features of gastric carcinomas with the CpG island methylator phenotype and an association with Epstein-Barr virus. Cancer 2006; 106:1467-79.

5. Hoon D S, Spugnardi M, Kuo C, Huang S K, Morton D L, Taback B. Profiling epigenetic inactivation of tumor suppressor genes in tumors and plasma from cutaneous melanoma patients. Oncogene 2004; 23:4014-22.

6. Mori T, O'Day S J, Umetani N, et al. Predictive utility of circulating methylated DNA in serum of melanoma patients receiving biochemotherapy. J Clin Oncol 2005; 23:9351-8.

7. Marini A, Mirmohammadsadegh A, Nambiar S, Gustrau A, Ruzicka T, Hengge U R. Epigenetic inactivation of tumor suppressor genes in serum of patients with cutaneous melanoma. J Invest Dermatol 2006; 126:422-31.

8. Spugnardi M, Tommasi S, Dammann R, Pfeifer G P, Hoon D S. Epigenetic inactivation of RAS association domain family protein 1 (RASSF1A) in malignant cutaneous melanoma. Cancer Res 2003; 63:1639-43.

9. Sierko E, Wojtukiewicz M Z, Kisiel W. The role of tissue factor pathway inhibitor-2 in cancer biology. Semin Thromb Hemost 2007; 33:653-9.

10. Nobeyama Y, Okochi-Takada E, Furuta J, et al. Silencing of tissue factor pathway inhibitor-2 gene in malignant melanomas. Int J Cancer 2007; 121:301-7.

11. Mazieres J, He B, You L, et al. Wnt inhibitory factor-1 is silenced by promoter hypermethylation in human lung cancer. Cancer Res 2004; 64:4717-20.

12. Urakami S, Shiina H, Enokida H, et al. Epigenetic inactivation of Wnt inhibitory factor-1 plays an important role in bladder cancer through aberrant canonical Wnt/beta-catenin signaling pathway. Clin Cancer Res 2006; 12:383-91.

13. Urakami S, Shiina H, Enokida H, et al. Wnt antagonist family genes as biomarkers for diagnosis, staging, and prognosis of renal cell carcinoma using tumor and serum DNA. Clin Cancer Res 2006; 12:6989-97.

14. Taniguchi H, Yamamoto H, Hirata T, et al. Frequent epigenetic inactivation of Wnt inhibitory factor-1 in human gastrointestinal cancers. Oncogene 2005; 24:7946-52.

15. Lin Y C, You L, Xu Z, et al. Wnt inhibitory factor-1 gene transfer inhibits melanoma cell growth. Hum Gene Ther 2007; 18:379-86.

16. Akiyama Y, Watkins N, Suzuki H, et al. GATA4 and GATA-5 transcription factor genes and potential downstream antitumor target genes are epigenetically silenced in colorectal and gastric cancer. Mol Cell Biol 2003; 23:8429-39.

17. Guo M, Akiyama Y, House M G, et al. Hypermethylation of the GATA genes in lung cancer. Clin Cancer Res 2004; 10:7917-24.

18. Toyota M, Ahuja N, Ohe-Toyota M, Herman J G, Baylin S B, Issa J P. CpG island methylator phenotype in colorectal cancer. Proc Natl Acad Sci USA 1999; 96:8681-6.

19. Van Rijnsoever M, Elsaleh H, Joseph D, McCaul K, lacopetta B. CpG island methylator phenotype is an independent predictor of survival benefit from 5-fluorouracil in stage III colorectal cancer. Clin Cancer Res 2003; 9:2898-903.

20. Balch C M, Buzaid A C, Soong S J, et al. Final version of the American Joint Committee on Cancer staging system for cutaneous melanoma. J Clin Oncol 2001; 19:3635-48.

21. Li L C, Dahiya R. MethPrimer: designing primers for methylation PCRs. Bioinformatics 2002; 18:1427-31.

22. Umetani N, de Maat M F, Mori T, Takeuchi H, Hoon D S. Synthesis of universal unmethylated control DNA by nested whole genome amplification with phi29 DNA polymerase. Biochem Biophys Res Commun 2005; 329:219-23.

### Example II

### Assessment of MINT 17 Methylation in Primary Breast Cancer and Normal Breast Epithelia

### Abstract

Background: Methylated-in-tumor loci (MINTs) are non-coding DNA sequences containing CpG islands. The aim of this study was to assess the presence of aberrant methylation of MINT 17 in primary breast cancer. We believed that MINT 17 methylation index is significantly higher in primary breast tumor tissue than in normal breast tissue.

Methods: Paraffin-embedded breast tissues of 42 patients were collected. This selection comprised 26 breast cancer and 16 non-breast cancer patients. DNA was isolated from primary tumor tissues, and normal breast epithelia DNA was isolated from non-cancer breast tissue. DNA was subjected to sodium bisulfate modification. Absolute quantitative assessment of methylated alleles (AQAMA) was performed to assess methylation status of MINT 17 by multiplex quantitative methylation-specific PCR. The results were expressed as methylation index (MI): MI=methylated copy number/(Methylated copy number+unmethylated copy number). Medians were compared using Wilcoxon rank sum test. Area under the Receiver Operating Characteristics curve was calculated using logistic regression.

Results: Median MI for MINT 17 was significantly higher in tumor tissue (0.059) than in normal breast epithelia (0); p=0.0001. MI did not exceed 0.03 in normal epithelia, whereas MI exceeded 0.03 in 77% of the cancer tissues. The AUC for MINT 17 methylation as a predictor for cancer was 0.887. There was no significant difference in median MI between stage I and IV cancer patients. In cancer patients, median MINT 17 MI was significantly higher in ER(+) (0.315) than in ER(−) patients (0.043); p=0.033. PR(+) patients had higher median MI (0.313) than PR(−) patients (0.053); p=0.055. The AUC for MINT 17 MI as predictor for ER status and PR status was 0.785 and 0.737, respectively.

Conclusion: MINT 17 MI is significantly higher in primary breast cancer than in normal, breast epithelia, and is an indicator for presence of cancer in breast tissue. MINT 17 MI is related to ER and PR status in primary breast tumors.

### Introduction

Aberrant DNA methylation is a key epigenetic event contributing to tumorigenesis of several solid malignancies, including breast cancer. In mammals, CpG dinucleotides form the prime target of methylation. CpGs are predominantly found in dense clusters in the promoter region of genes. Hypermethylation of promoter regions leads to transcriptional silencing of the affected gene, and may compromise expression of tumor-related genes. Methylated-in-tumor loci (MINTs) are non-coding DNA sequences containing CpG islands. In colorectal cancer, methylation of MINTs is shown to concur with gene promoter hypermethylation, and is associated with malignant transformation and disease progression. In breast cancer, assessment of methylation of MINTs may be a valuable surrogate marker for prognostic variables. The aim of this study was to assess the presence of aberrant methylation of MINT 17 in primary breast cancer. We believed that MINT 17 methylation index is significantly higher in primary tumor than in normal breast tissue.

### Patients and Methods

Patients: Formalin-fixed paraffin-embedded (FFPE) breast tissues of 42 patients were retrospectively collected. This selection included 26 breast cancer and 16 non-cancer patients. Breast cancer patients were diagnosed with either AJCC stage I or IV breast cancer of the invasive ductal type (n=10 and n=16, respectively) (Table 1). All patients without a history of breast cancer had undergone breast reduction surgery. Breast tissues of these patients were reviewed by a pathologist and judged negative for malignancy, atypia, or benign proliferative lesions.

TABLE 1
Cancer patient characteristics

| | | n = 26 |
|---|---|---|
| Age | Mean (SD) | 62.9 (14.4) |
| | Range | 37-89 |
| Stage | I | 10 |
| | IV | 16 |
| ER | Negative | 9 |
| | Positive | 15 |
| | missing | 1 |
| PR | Negative | 13 |
| | Positive | 12 |
| | missing | 1 |
| HER2 | Negative | 16 |
| | Positive | 8 |
| | missing | 2 |
| LVI | No lymphovascular invasion | 13 |
| | Lymphovascular invasion | 12 |
| | missing | 1 |
| Histologic Grade | Well differentiated | 7 |
| | Moderately differentiated | 6 |
| | Poorly differentiated | 12 |
| | missing | 1 |
| S-phase fraction | Low | 6 |
| | Intermediate | 6 |
| | High | 10 |
| | missing | 4 |
| Ploidy | Not diploid | 18 |
| | Diploid | 5 |
| | missing | 3 |
| Ki67 | Low | 10 |
| | Intermediate | 7 |
| | High | 8 |
| | missing | 1 |

Tissue preparation: Tissue sections of 8 μm thickness were cut for each specimen and mounted on non-coated slides. Tissue was harvested from slides by microscope-assisted needle microdissection, using an H&E slide as reference. Dissected tissues were incubated at 50° C. for 16 hours in 50 μl lysis buffer containing 2.5% TWEEN 20 and 2.4 mAU proteinase K, followed by heat inactivation of proteinase K enzyme at 95° C. for 10 minutes. After purification with phenol-chloroform-isoamyl alcohol and precipitation with ethanol, the DNA purity was measured with UV spectrophotometry, and the dsDNA quantity was determined with the PICOgreen assay. One μg of DNA was treated with sodium bisulfate/hydroquinone to achieve conversion of unmethylated cytosines to uracil.

AQAMA assay: Quantitative assessment of methylated alleles (AQAMA) was designed to perform quantitative real-time PCR using one single set of primers for PCR amplification of both methylated and unmethylated alleles. Sequence-specific MGB probes were used to differentiate between amplification of methylated and unmethylated DNA. Labeling of these probes with FAM and VIC dyes respectively allowed for simultaneous detection of both amplification reactions. Separate plasmid standard dilution series of known copy numbers were used to quantify methylated and unmethylated amplicons. SssI methylase-treated lymphocyte DNA was used as a positive methylated control, and lymphocyte DNA amplified with Φ29 DNA polymerase served as an unmethylated control. PCR was performed in a 384-well plate using the ABI Prism 7900HT Sequence Detection System. All sample reactions were run in triplicate.

Analysis: For each sample, methylation index (MI) was calculated as follows: MI=methylated copy number/(methylated copy number+unmethylated copy number). To assess differences in methylation index, medians were compared using Wilcoxon rank sum test for nominal data and Kruskal-Wallis test for ordinal data. Area under the Receiving Operator Characteristics curve (AUC) was calculated using logistic regression. Statistical calculations were performed using SAS software version 8.02.

### Conclusions

MINT 17 MI is significantly higher in primary breast cancer than in normal breast epithelia, and is a predictor for the presence of cancer in breast tissue. In primary breast tumors, methylation of MINT 17 is associated with ER and PR expression, and is related to low S-phase fraction. Our results suggest that methylation of MINT 17 is an epigenetic event occurring exclusively in the context of malignant transformation. In particular, DNA methylation may be an important factor in the development of hormone receptor positive breast cancer.

### REFERENCES

1. de Maat M F, van de Velde C J, van der Werff M P, et al. Quantitative analysis of methylation of genomic loci in early-stage rectal cancer predicts distant recurrence. J Clin Oncol 26(14):2327-35, 2008.

2. de Maat M F, Umetani N, Sunami E, et al. Assessment of methylation events during colorectal tumor progression by absolute quantitative analysis of methylated alleles. Mol Cancer Res 5(5):461-71, 2007.

### Example III

### Absolute Quantitative Assessment of Methylated Alleles in Breast Cancer

### Objectives:

To determine the value of quantification of DNA methylation in predicting survival and prognosis of patients with infiltrative ductal carcinoma of the breast, using a panel of 4 DNA markers.
To assess presence of a “methylator phenotype” (i.e., can patients be clustered in meaningful groups according to the methylation status of these 4 markers?). To study potential relation of a methylator phenotype in biological behavior and molecular subtypes of breast cancer.
To relate methylation markers to tumor/pathological characteristics.

### Background:

There is increasing evidence that breast cancer is a heterogeneous disease comprising separate molecular subtypes; it has been proposed that breast cancer may in fact be a collection of several distinct diseases. Current models for staging and classification fall short in accurately predicting disease behavior and outcome. Moreover, failure of current models to predict the need for, and response to, therapy leads to both undertreatment and overtreatment of patients. Better understanding of molecular origins of the diverse subtypes of breast cancer may help overcome this problem.

The aim of this study was to assess usefulness of a DNA methylation marker panel in predicting disease outcome and distinguishing molecular subtypes of breast cancer. DNA methylation refers to the binding of a methyl group (CH₃) to the “C” of “CG” dinucleotides in genomic DNA. CH₃-tagging of CGs in the promoter region of a gene leads to transcriptional repression or silencing of the tagged gene. DNA methylation is a major effector of regulation of DNA transcription, responsible for physiological processes such as X-chromosome inactivation and genomic imprinting. However, aberrant methylation of genes crucial to normal cell function contributes to the development of several types of cancer, including breast cancer. DNA methylation of genes may be a valuable marker for disease behavior and outcome in two ways; it is conceivable that methylation, and thus silencing, of genes significant for normal cell behavior may relate to outcome, according to the functions and associated pathways of the gene in question. Secondly, it has been demonstrated in several cancer types that DNA hypermethylation is not randomly or evenly distributed amongst cases, but goes hand in hand with certain molecular subtypes. This has been studied most extensively in colorectal tumors, where a “methylator phenotype” is related to a MSI-H, BRAF-mutation-high phenotype. This may equally be the case in breast cancer, where some studies indicate that aberrant methylation occurs in ER positive patients rather than in ER negative patients. However, few studies have been conducted to quantitatively assess methylation to predict the breast cancer outcome and presence of a “methylator” subtype, and its possible relation to postulated molecular subtypes (i.e., normal-like, luminal A, luminal B, basal-like, ER-/HER2+).

### Markers:

Methylation markers used in this study were MINT17, MINT31, RARβ2 and RASSF1A.

MINTs are non-coding DNA loci of which hypermethylation is shown to concur with global promoter hypermethylation in colorectal cancer. We conducted a pilot study of MINTs in normal breast epithelia and breast cancer, demonstrating that two MINT loci (MINT17 and MINT31) are highly methylated in breast cancer but not in normal epithelia. In cancer patients, methylation of these MINTs were related to hormone receptor status and stage progression. MINT17 and MINT31 are novel markers in breast cancer.

RARβ2 and RASSF1A are established methylation markers in breast cancer. RASSF1A (RAS associated domain family 1A) is a tumor suppressor gene acting by blocking oncogene-mediated c-Jun kinase activation. It may also have a role in maintaining genomic stability. Previous studies show that hypermethylation of the promoter region is reversely related to protein expression. Methylation of RASSF1A in breast cancer is related to ER expression. RARβ2 (retinoic acid receptor beta 2) is a putative tumor suppressor gene and its promoter hypermethylation is related to breast cancer metastasis.

### Tissue:

The JANE-series is a retrospective series of patients diagnosed with stage 1-3 invasive breast cancer and treated at Leiden University Medical Center between 1985 and 1995. For this study, a selection was made of patients with infiltrative ductal carcinoma, while maintaining chronological order as apparent from study ID#. Patients with a diagnosis of lobular cancer or mucinous cancer, and patients with insufficient volume of available paraffin tumor tissue were excluded. The final selection comprised 384 patients.

### Methods:

Tumor tissue was harvested by needle microdissection from 3×8 μm deparrafinized paraffin sections for each patient. Isolated tissue was incubated with proteinase K lysis buffer, and DNA was purified. DNA was then modified using the Epitect Qiagen bisulfate modification kit, resulting in C to T conversion of all unmethylated C bases while preserving methylated C's, hence achieving different sequences for methylated and unmethylated genome. Quantitative real-time PCR was performed according to the “AQAMA” protocol, using a single “common sequence” primer set for methylated and unmethylated reactions, with specific probes for both methylated and unmethylated sequences. This multiplex method allows for absolute quantification of methylated alleles, using plasmid standard dilutions for both methylated and unmethylated sequences to determine copy numbers (cn) for both reactions. For each locus, a methylation index (MI) is derived by dividing the methylated (M) copy number by the total copy numbers: MI=Mcn/(Mcn+Ucn).

All samples and reactions were performed as triplicates. Controls for each assay included universal methylated control (UMC; SSS1-treated PBL DNA), universal unmethylated control (UUC, phi-29-treated PBL DNA), methylated cell line DNA (SNU), unmethylated cell line DNA (FN-001), untreated PBL, H₂O and blanks.

Methylation status was also assessed for 16-35 normal breast epithelia samples of patients without a history of breast cancer. Mean MI and standard deviation were calculated for these samples, and are as follows:

| | Mean MI | SD | Mean + 2SD |
|---|---|---|---|
| MINT17 | 0.004031 | 0.00850 | 0.0210 |
| MINT31 | 0.004280 | 0.01594 | 0.0362 |
| RARβ2 | 0.000879 | 0.00325 | 0.0074 |
| RASSF1A | 0.103361 | 0.16611 | 0.4356 |

Data for normal breast samples were included in a separate SPSS file.

### Data Format:

Data were expressed as:
Methylation Index (MI); continuous value between 0.0 and 1.0
Labeled “MINT17_MI”
Binary score derived from methylation index: methylation, YES or NO
0=MI is zero
1=MI is larger than zero
Labeled “MINT17_BIN”
Binary score derived from methylation index of normal breast samples: MI exceeds mean value+2 standard deviations in normal breast epithelia, YES or NO
0=MI does not exceed mean+2SD [normal breast]
1=MI is equal to or exceeds mean+2SD [normal breast]
Labeled “MINT17_{—}2SD”

### Statistical Analysis:

Points of interest for statistical analysis included:

1. Relation between each marker and clinicopathologic parameters such as ER/PR/HER2neu status, ki-67, AJCC stage, presence of lymph node metastatis, histologic grade, and, if available, lymphovascular invasion, S-phase % and MAI.

2. Combined markers for prediction of clinicopathologic features. Is a marker panel useful (any combination of markers) (i.e., for prediction of stage progression or molecular subtype)?

3. Cluster analysis according to methylation status of these markers. Can outcome be predicted according to methylation characteristics of the tumor?

4. Cluster analysis according to methylation status of the markers and ER, PR, HER2 status (if CKs available).

5. Is methylation related to age?

6. Survival analysis for each marker, looking at OS (disease related) and recurrence (distant, regional, local, locoregional).

7. Survival analysis per stratum: AJCC stage, ER status, LN+ vs LN−.

8. Stratified per treatment; for Hormonal Therapy in particular (patients treated with hormonal therapy, n=55/384 according to the database; type of HT not listed).

9. If markers are predictive for distant recurrence, is this for a particular site/organ system (i.e., bone/liver/lung/CNS)?

10. Survival analysis for markers combined (is there a meaningful combination of markers/marker panel?).

11. Multivariate analysis.

12. Is quantification of methylation relevant? Does the actual value of methylation index add to predictive power as compared to methylation status expressed binary?

13. Are there meaningful cut-off points for MI other than “Normal epithelia [mean+2SD]” or “Methylation YES or N”?

14. Is there a relation between methylation of one marker and another? Is there any support for a methylator phenotype discernible, and if so, does this category of patients demonstrate differences in survival or clinicopathological features?

### Results:

Relation was analyzed between the Methylation Index for individual markers and clinicopathologic parameters.

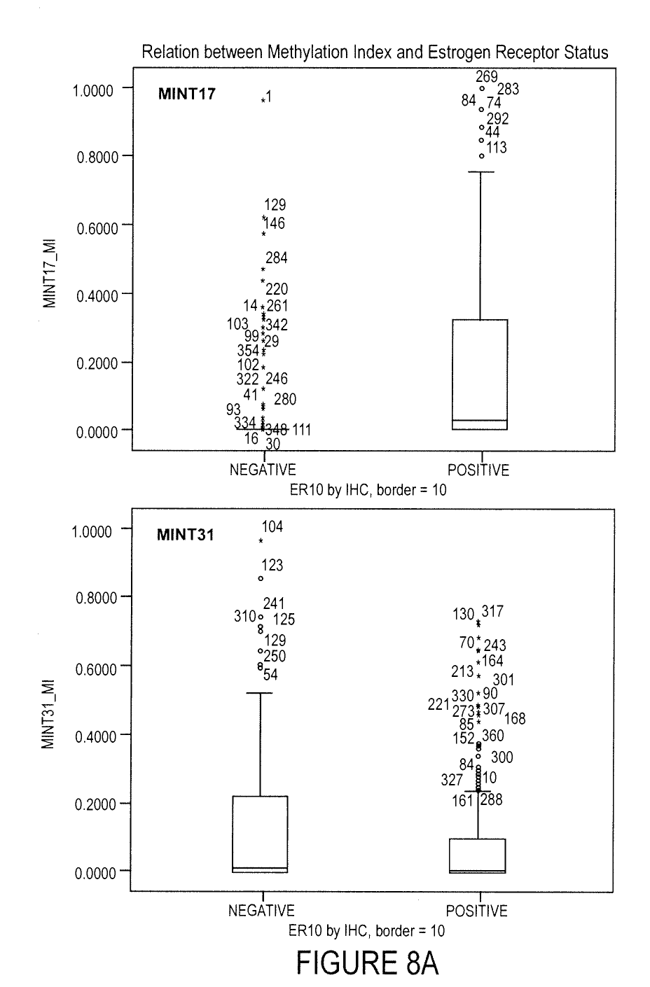

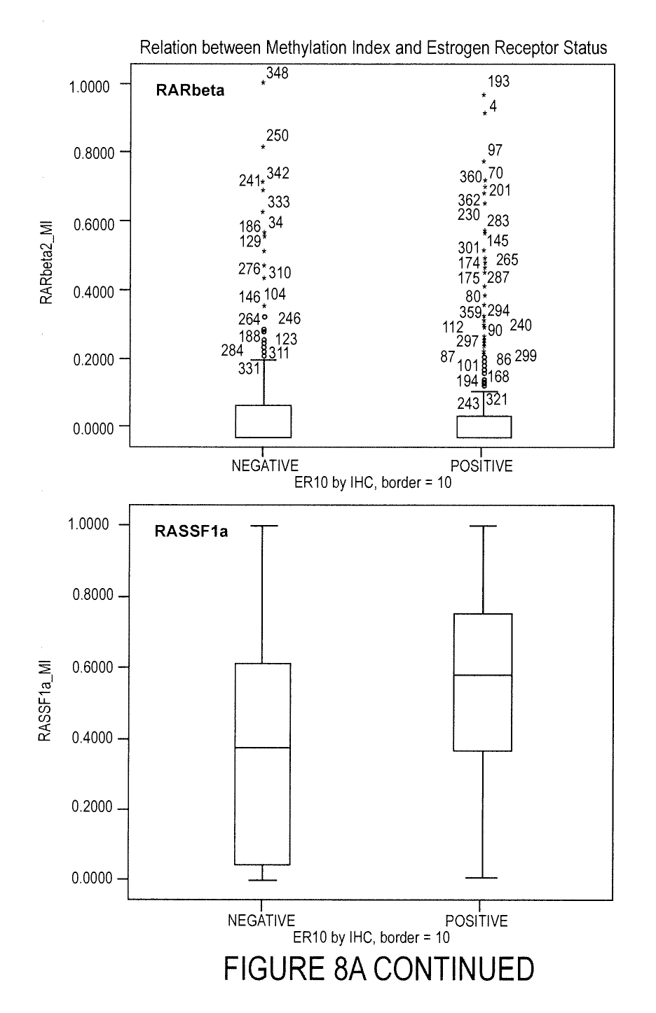

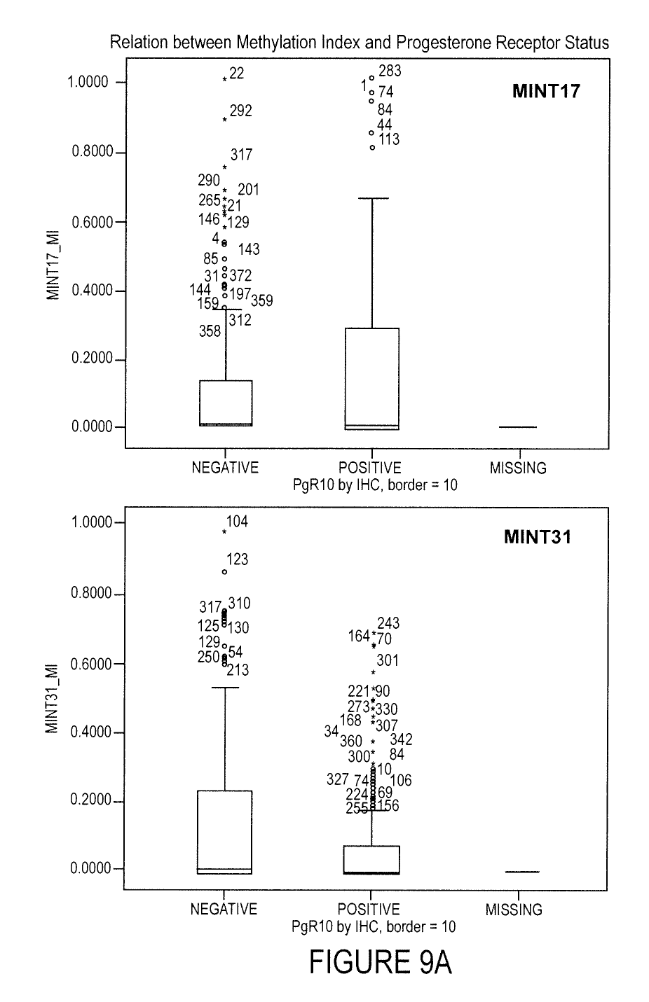

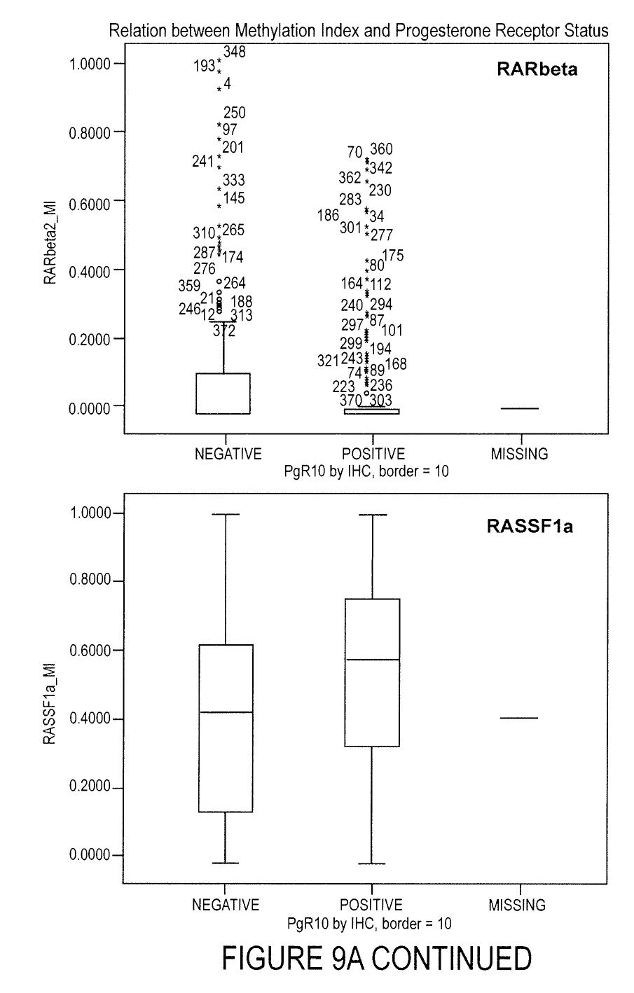

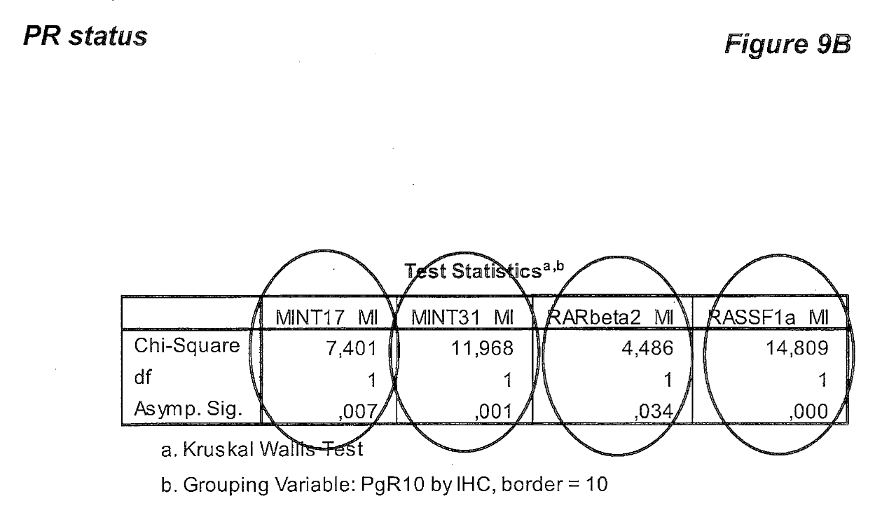

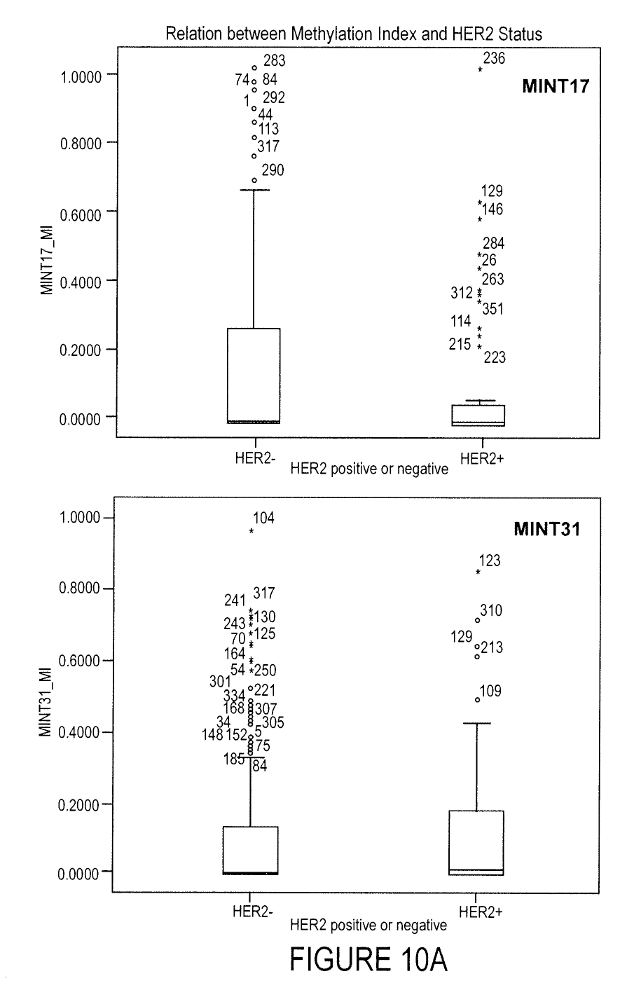

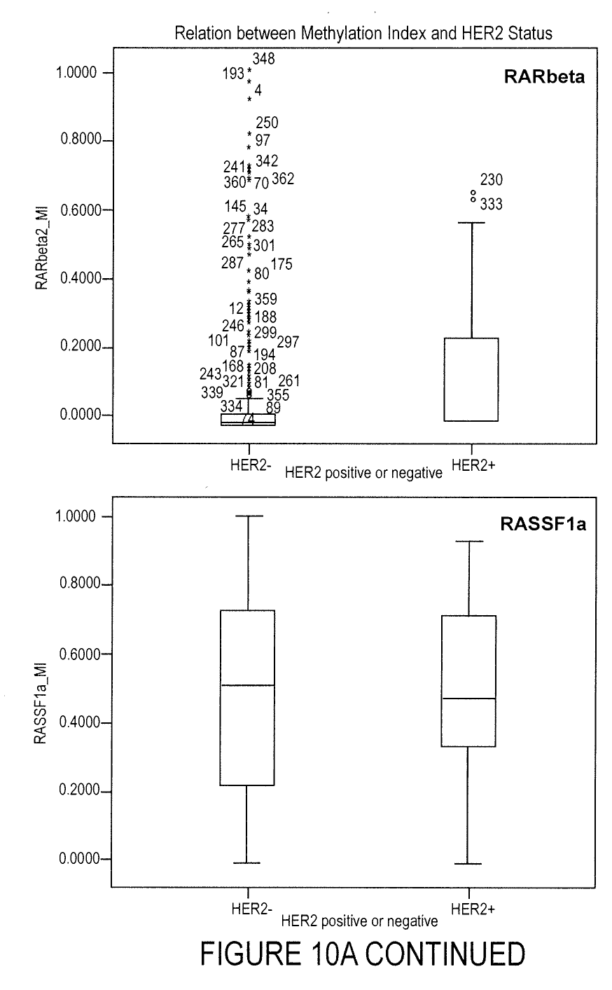

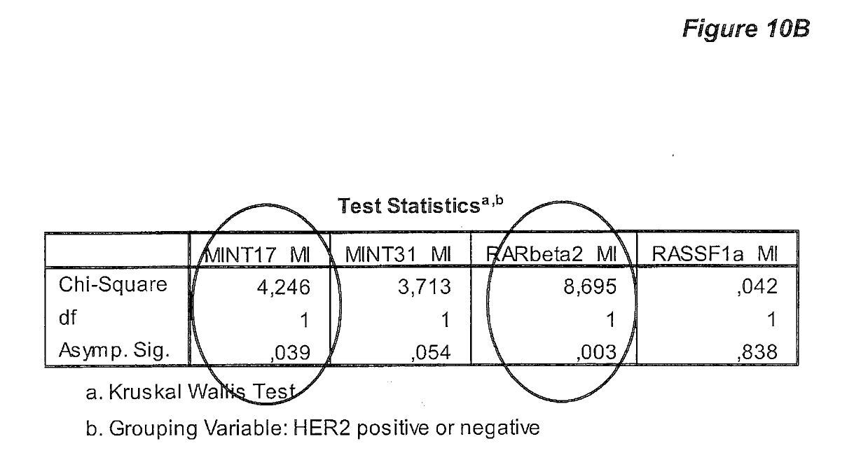

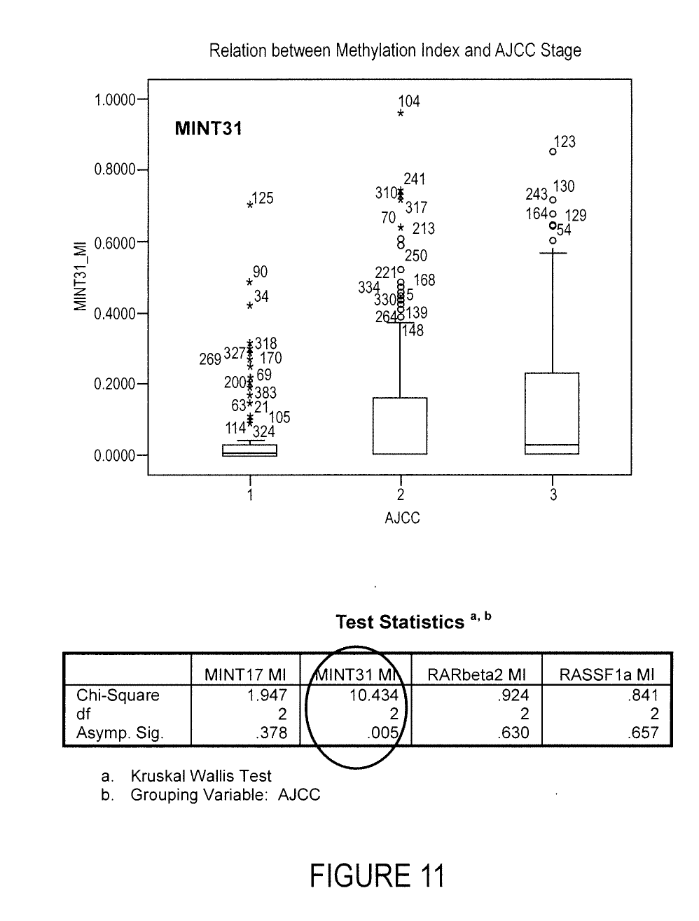

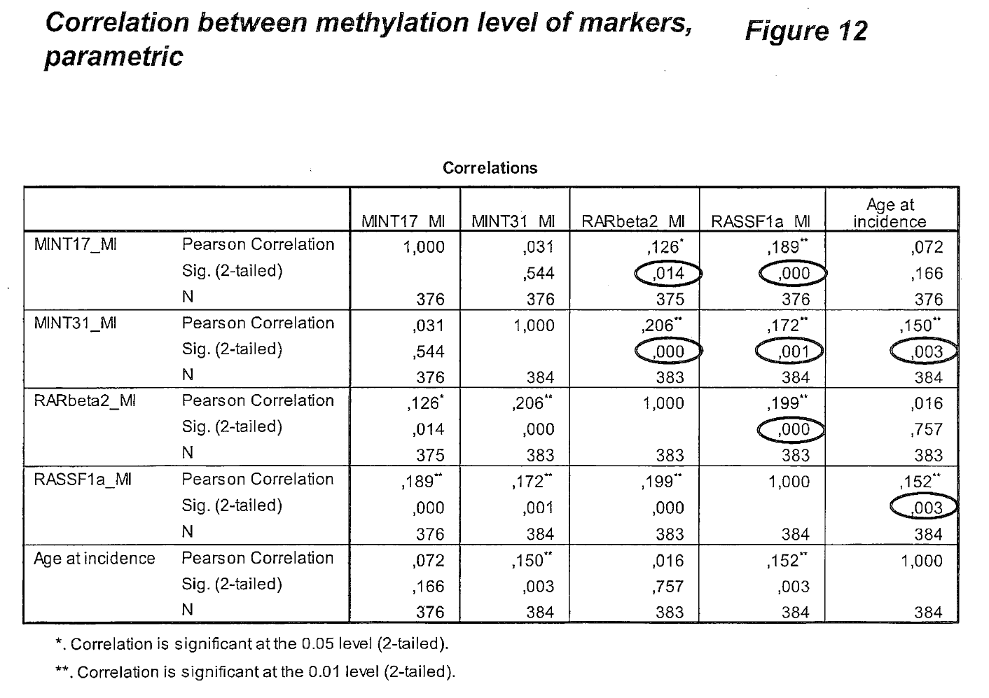

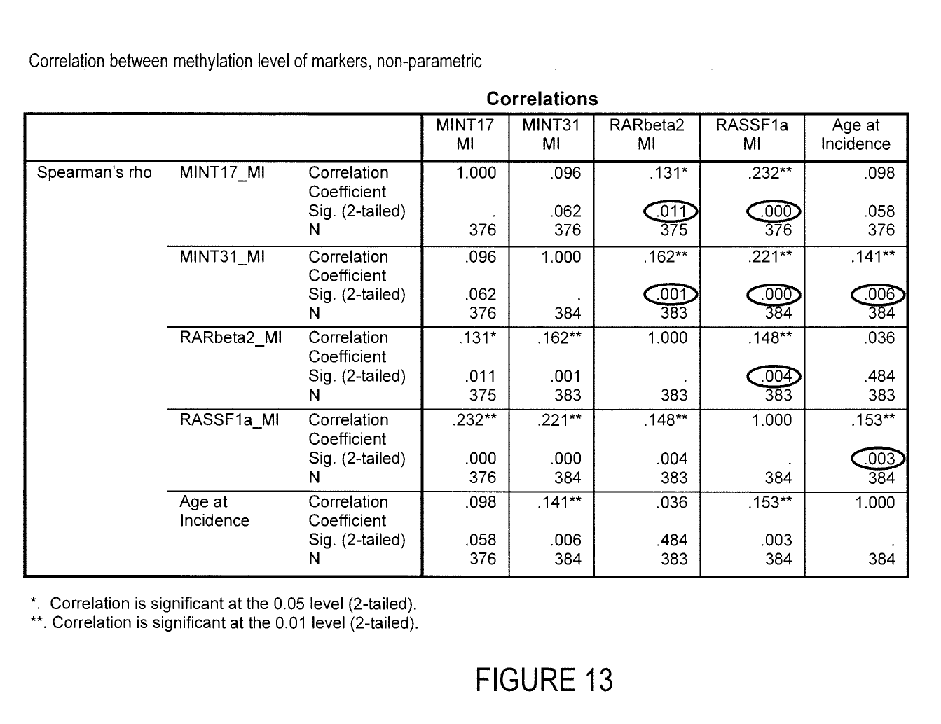

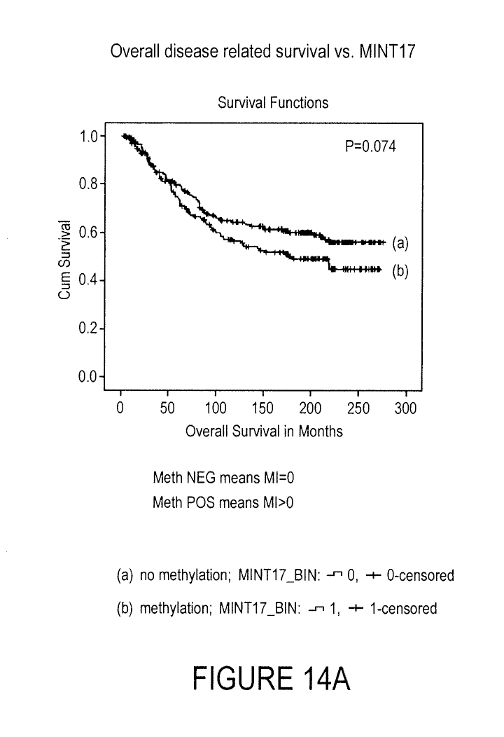

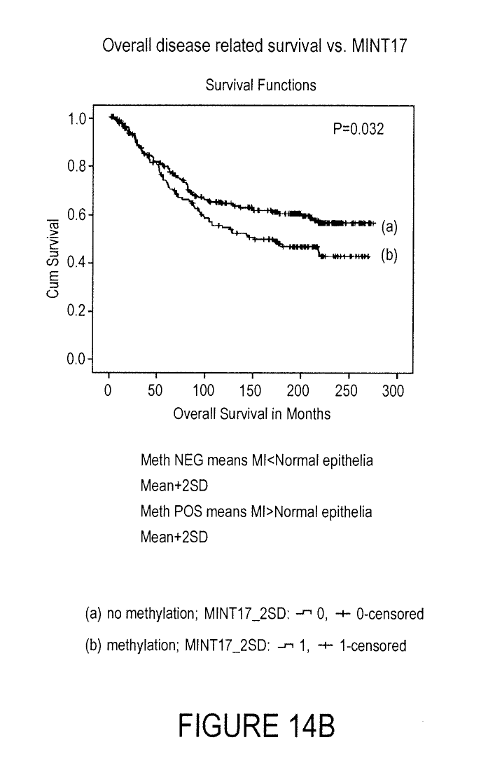

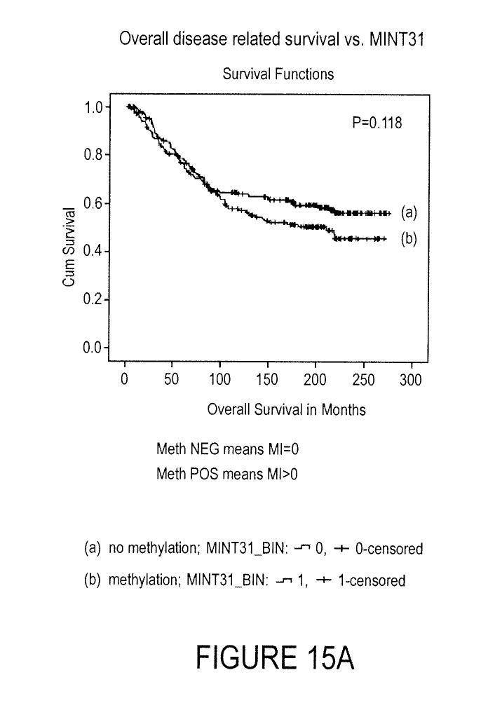

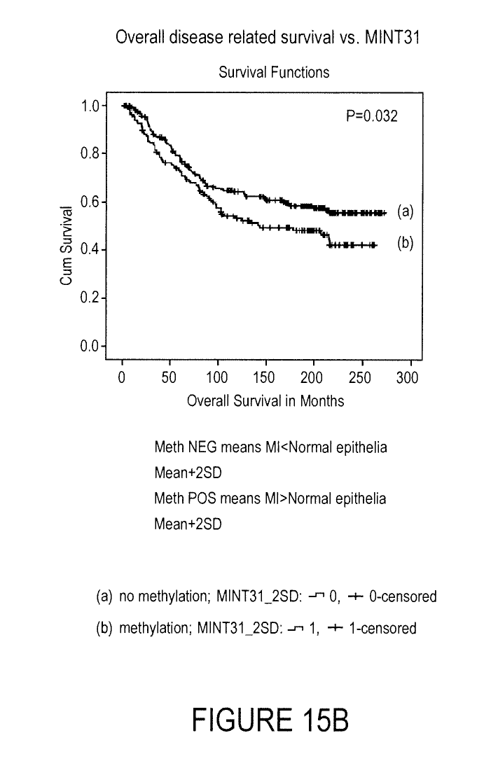

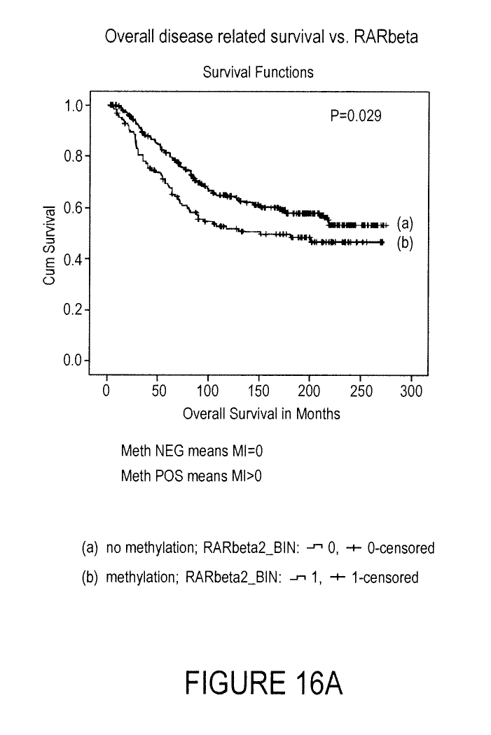

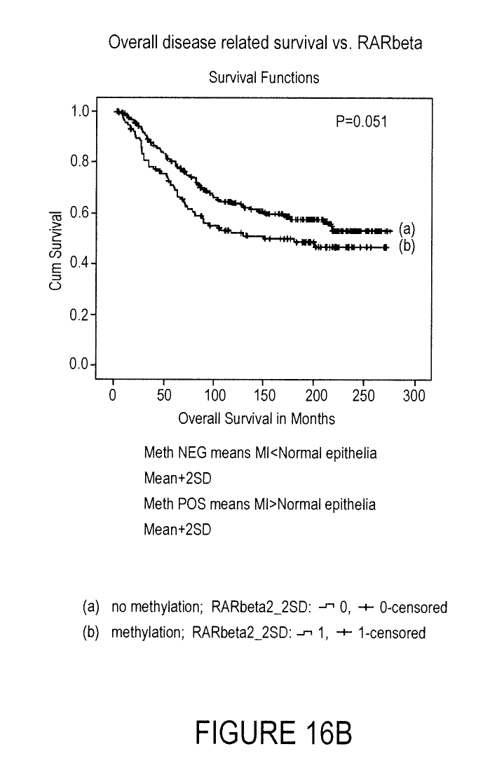

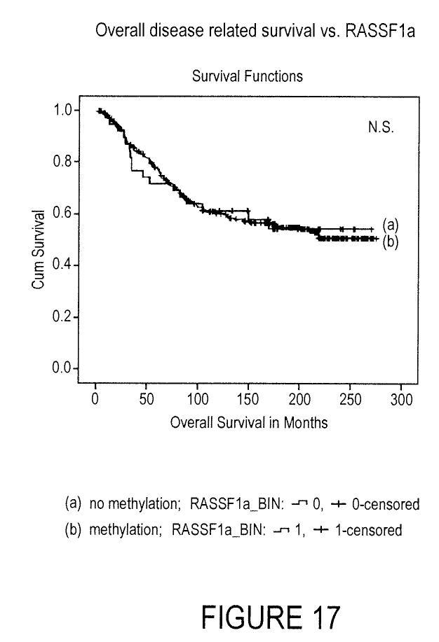

FIGS. 8-17 show the relation between methylation of the 4 markers and
Estrogen receptor status
Progesterone receptor status
HER2 status
AJCC stage

### Conclusions:

Methylation status of MINT17, MINT31, RARβ2 and RASSF1A is related to ER, PR, and HER2 status in breast cancer.
MINT31 is related to stage progression.
MINT17, MINT31 and RARβ2 predict disease related survival in patients with breast cancer.
Methylation of MINT17, MINT31 and RARβ2 is prognostically unfavorable.

All publications cited herein are incorporated by reference in their entirety.

What is claimed is:

1. A method of determining breast cancer status, comprising:
providing from a subject a sample containing breast cancer cells; and
determining the level of DNA methylation in MINT17, MINT31, or the promoter region of RASSF1A or RARβ2 (retinoic acid receptor beta 2) in the breast cancer cells,
wherein the level of DNA methylation in MINT17, MINT31, or the promoter region of RASSF1A or RARβ2 in the breast cancer cells, if higher than that in normal cells, indicates that the breast cancer is likely to be an aggressive breast cancer.

2. The method of claim 1, wherein the breast cancer is a primary or metastatic cancer.

3. The method of claim 1, wherein a higher level of DNA methylation in MINT17 or the promoter region of RASSF1A, or a lower level of DNA methylation in MINT31 in the breast cancer cells indicates that the breast cancer is more likely to be positive for ER (estrogen receptor).

4. The method of claim 1, wherein a higher level of DNA methylation in MINT17 or the promoter region of RASSF1A, or a lower level of DNA methylation in MINT31 or the promoter region of RARβ2 in the breast cancer cells indicates that the breast cancer is more likely to be positive for PR (progesterone receptor).

5. The method of claim 1, wherein a higher level of DNA methylation in the promoter region of RARβ2, or a lower level of DNA methylation in MINT17 in the breast cancer cells indicates that the breast cancer is more likely to be positive for HER2 (human epidermal growth factor receptor 2).

6. The method of claim 1, wherein a higher level of DNA methylation in MINT31 in the breast cancer cells indicates that the subject is likely to be suffering from a more aggressive breast cancer.

7. A method of predicting the outcome of breast cancer, comprising:
providing from a subject a sample containing breast cancer cells; and
determining the level of DNA methylation in MINT17, MINT31, or the promoter region of RARβ2 in the breast cancer cells,
wherein a higher level of methylation in MINT17, MINT31, or the promoter region of RARβ2 in the breast cancer cells indicates a less likelihood of overall survival.

8. A method of detecting breast cancer in a subject, comprising:
providing a DNA sample from a subject; and
determining the level of DNA methylation in MINT17 or MINT31 in the sample,
wherein the level of DNA methylation in MINT17 or MINT31 in the sample, if higher than that in normal cells, indicates that the subject is suffering from breast cancer.

9. The method of claim 8, wherein the breast cancer is an aggressive breast cancer.

10. The method of claim 8, wherein the breast cancer is a primary or metastatic cancer.


SEQUENCE LISTING (34 sequences)

SEQ ID NO: 1 - DNA, 20 bp, Artificial Sequence
  Synthetic oligonucleotide
gggcgttttattgggcgtat

SEQ ID NO: 2 - DNA, 20 bp, Artificial Sequence
  Synthetic oligonucleotide
aacctaaacgaccgccactt

SEQ ID NO: 3 - DNA, 20 bp, Artificial Sequence
  Synthetic oligonucleotide
gggtgttttattgggtgtat

SEQ ID NO: 4 - DNA, 21 bp, Artificial Sequence
  Synthetic oligonucleotide
aacctaaacaaccaccactta

SEQ ID NO: 5 - DNA, 21 bp, Artificial Sequence
  Synthetic oligonucleotide
tttcgtataaagcgggtattc

SEQ ID NO: 6 - DNA, 20 bp, Artificial Sequence
  Synthetic oligonucleotide
acgacccgctaaacaaaacg

SEQ ID NO: 7 - DNA, 23 bp, Artificial Sequence
  Synthetic oligonucleotide
ggatgtttgttttgtataaagtg

SEQ ID NO: 8 - DNA, 23 bp, Artificial Sequence
  Synthetic oligonucleotide
aaacatccaaaaaaacacctaac

SEQ ID NO: 9 - DNA, 20 bp, Artificial Sequence
  Synthetic oligonucleotide
gtgttaacgcgttgcgtatc

SEQ ID NO: 10 - DNA, 21 bp, Artificial Sequence
  Synthetic oligonucleotide
aaccccgcgaactaaaaacga

SEQ ID NO: 11 - DNA, 21 bp, Artificial Sequence
  Synthetic oligonucleotide
tttggttggagtgtgttaatg

SEQ ID NO: 12 - DNA, 23 bp, Artificial Sequence
  Synthetic oligonucleotide
caaaccccacaaactaaaaacaa

SEQ ID NO: 13 - DNA, 19 bp, Artificial Sequence
  Synthetic oligonucleotide
gaacgcgagcgattcgagt

SEQ ID NO: 14 - DNA, 19 bp, Artificial Sequence
  Synthetic oligonucleotide
gaccaatccaaccgaaacg

SEQ ID NO: 15 - DNA, 21 bp, Artificial Sequence
  Synthetic oligonucleotide
ggattgggatgttgagaatgt

SEQ ID NO: 16 - DNA, 21 bp, Artificial Sequence
  Synthetic oligonucleotide
caaccaatccaaccaaaacaa

SEQ ID NO: 17 - DNA, 20 bp, Artificial Sequence
  Synthetic oligonucleotide
tcgttcgtacgtcgattatc

SEQ ID NO: 18 - DNA, 21 bp, Artificial Sequence
  Synthetic oligonucleotide
aaaaaaatacccacgaactcg

SEQ ID NO: 19 - DNA, 25 bp, Artificial Sequence
  Synthetic oligonucleotide
tattttgtttgtatgttgattattg

SEQ ID NO: 20 - DNA, 21 bp, Artificial Sequence
  Synthetic oligonucleotide
aaactcaacacacaaccactc

SEQ ID NO: 21 - DNA, 24 bp, Artificial Sequence
  Synthetic oligonucleotide
tatagcgaatttaatcgattttcg

SEQ ID NO: 22 - DNA, 20 bp, Artificial Sequence
  Synthetic oligonucleotide
gactacacctccgctaaacg

SEQ ID NO: 23 - DNA, 30 bp, Artificial Sequence
  Synthetic oligonucleotide
tgggtattatagtgaatttaattgattttt

SEQ ID NO: 24 - DNA, 23 bp, Artificial Sequence
  Synthetic oligonucleotide
cccaactacacctccactaaaca

SEQ ID NO: 25 - DNA, 21 bp, Artificial Sequence
  Synthetic oligonucleotide
aggggttaggttgaggttgtt

SEQ ID NO: 26 - DNA, 20 bp, Artificial Sequence
  Synthetic oligonucleotide
taaagtgaggggtggtgatg

SEQ ID NO: 27 - DNA, 21 bp, Artificial Sequence
  Synthetic oligonucleotide
tctacctcttcccaaattcca

SEQ ID NO: 28 - DNA, 21 bp, Artificial Sequence
  Synthetic oligonucleotide
aaaaacacttccccaacatct

SEQ ID NO: 29 - DNA, 15 bp, Artificial Sequence
  Synthetic oligonucleotide
ttggatggatcgcgg

SEQ ID NO: 30 - DNA, 16 bp, Artificial Sequence
  Synthetic oligonucleotide
aggtttcgtcgtgttt

SEQ ID NO: 31 - DNA, 19 bp, Artificial Sequence
  Synthetic oligonucleotide
tattttggatggattgtgg

SEQ ID NO: 32 - DNA, 18 bp, Artificial Sequence
  Synthetic oligonucleotide
aggttttgttgtgtttat

SEQ ID NO: 33 - DNA, 10 bp, Artificial Sequence
  Synthetic oligonucleotide
agttgagcga

SEQ ID NO: 34 - DNA, 10 bp, Artificial Sequence
  Synthetic oligonucleotide
agttgagtga